(19) United States
(12) Patent Application Publication
(10) Pub. No.: US 2023/0330368 A1
(43) Pub. Date: Oct. 19, 2023

(54) REPLACEABLE FILTER SYSTEMS FOR MECHANICAL VENTILATION

(71) Applicant: Covidien LP, Mansfield, MA (US)

(72) Inventors: Matthew J. PHILLIPS, Carlsbad, CA (US); Amand V. KASIMATIS, Carlsbad, CA (US); William F. FLANAGAN, San Diego, CA (US); Mark H. OGILVIE, Encinitas, CA (US)

(73) Assignee: Covidien LP, Mansfield, MA (US)

(21) Appl. No.: 18/296,049

(22) Filed: Apr. 5, 2023

Related U.S. Application Data
(60) Provisional application No. 63/330,578, filed on Apr. 13, 2022.

(51) Int. Cl. A61M 16/00; A61M 16/10

(52) U.S. Cl. CPC A61M 16/0063; A61M 16/105; A61M 2016/0042

(57) ABSTRACT

Systems and methods for filtration of expiratory gases are disclosed. In an example, the technology relates to a method for replacing expiratory filters of a ventilation system without breaking a breathing circuit. The method may include opening a first channel and closing a second channel such that expiratory gases flow through a first filter coupled to the first channel; closing the first channel and opening the second channel such that the expiratory gases flow through a second filter coupled to the second channel; and while the expiratory gases are flowing through the second channel, replacing filter media of the first filter while maintaining pressure in the breathing circuit.

### CROSS-REFERENCE TO RELATED APPLICATIONS

This application claims the benefit of U.S. Provisional Application No. 63/330,578 filed Apr. 13, 2022, titled “Replaceable Filter Systems for Mechanical Ventilation,” which is incorporated by reference in its entirety. To the extent appropriate a claim of priority is made to the above-mentioned application.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. The conduit or tubing may form what is known as a patient circuit. The pressurized gas flows to the patient through an inhalation or inspiratory side or limb of the patient circuit. Gases exhaled by the patient then flow back to the ventilator through an exhalation or expiratory side or limb of the patient circuit.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for filtering exhaled gases. In an aspect, the technology relates to a ventilation system. The ventilation system includes an inspiratory limb that carries breathing gases from a medical ventilator towards a patient; an expiratory limb that carries expiratory gases from the patient towards the medical ventilator; and a dual-channel expiratory filtration system coupled to the expiratory limb and configured to receive unfiltered expiratory gases carried by the expiratory limb. The dual-channel expiratory filtration system includes an inlet that receives the unfiltered expiratory gases from the expiratory limb; a first channel, pneumatically coupled to the inlet, including a first filter that filters the unfiltered expiratory gases to form filtered expiratory gases; a second channel, pneumatically coupled to the inlet, including a second filter that filters the unfiltered expiratory gases to form filtered expiratory gases; at least one valve, wherein when the valve is in a first state, the unfiltered expiratory gases from the inlet flow through the first channel, and when the valve is in a second state, the unfiltered expiratory gases from the inlet flow through the second channel; and an outlet, pneumatically coupled to the first channel and the second channel, that carries the filtered expiratory gases towards the ventilator.

In an example, the at least one valve includes a first valve positioned in the first channel and a second valve positioned in the second channel. In another example, the at least one valve is a rotatable valve that is: coupled to the inlet; selectively coupled to the first channel when the rotatable valve is in the first state; and selectively coupled to the second channel when the rotatable valve is in the second state. In a further example, the valve is controlled by the ventilator. In yet another example, the valve is configured to be controlled manually by a clinician. In still another example, the system further includes a first check valve in a first post-filter segment of the first channel; and a second check valve in a second post-filter segment of the second channel. In still yet another example, the system further includes a sterilization system configured to sterilize the first filter.

In another aspect, the technology relates to a method for filtering expiratory gases while maintaining pressure in a breathing circuit. The method includes receiving an unfiltered expiratory gas flow through a first filter positioned in an expiratory limb of breathing circuit; receiving a selection to change the unfiltered expiratory gases to flow through a second filter positioned in the expiratory limb of the breathing circuit; and subsequent to receiving the selection, receiving the unfiltered expiratory gas flow through the second filter, but not the first filter, while maintaining pressure in the breathing circuit.

In an example, the method further includes, while the unfiltered expiratory gas flow is received through the second filter, sterilizing the first filter. In another example, the method further includes receiving a selection to change the unfiltered expiratory gases to flow through the first filter positioned in the expiratory limb of the breathing circuit; and receiving the unfiltered expiratory gas flow through the first filter, but not the second filter, while maintaining pressure in the breathing circuit. In yet another example, the method further includes, while the unfiltered expiratory gas flow is received through the first filter, sterilizing the second filter. In still another example, the first filter is positioned in a first channel of a filtration system in the expiratory limb, and the second filter is positioned in a second channel of the filtration system.

In another aspect, the technology relates to a method for replacing expiratory filters of a ventilation system without breaking a breathing circuit. The method includes opening a first channel and closing a second channel such that expiratory gases flow through a first filter coupled to the first channel; closing the first channel and opening the second channel such that the expiratory gases flow through a second filter coupled to the second channel; and while the expiratory gases are flowing through the second channel, replacing filter media of the first filter while maintaining pressure in the breathing circuit.

In an example, the first channel and the second channel are both coupled to an inlet and an outlet. In another example, the method further includes subsequent to replacing the filter media of the first filter: reopening the first channel and reclosing the second channel such that the expiratory gases flow through first channel and the first filter; and while the expiratory gases are flowing through the first channel, replacing filter media of the second filter. In another example, the method further includes subsequent to closing the first channel and opening the second channel, and prior to replacing the filter media of the first filter, sterilizing the first filter. In still another example, opening the first channel and closing the second channel comprises rotating a rotatable valve. In yet another example, opening the second channel and closing the first channel comprises rotating the rotatable valve. In a further example, closing the second channel comprises compressing tubing of the second channel. In still yet another example, opening the first channel and closing the second channel includes rotating a rotatable valve.

In another example, the technology relates to a method for controlling a dual-channel expiratory filtration system for replacement of filters during ventilation. The method includes positioning a channel-selection mechanism of the dual-channel expiratory filtration system in a first state to allow for expiratory gases to flow through a first channel and a first filter and to prevent the expiratory gases from flowing through a second channel and a second filter; positioning the channel-selection mechanism in a second state to allow for expiratory gases to flow through the second channel and the second filter and to prevent the expiratory gases from flowing through the first channel and the first filter; and while the channel-selection mechanism is in the second state, activating a sterilization system to sterilize the first filter.

In an example, the method is performed by a medical ventilator. In another example, the method further includes locking a filter housing of the first filter prior to completion of the sterilization of the first filter; presenting a sterilization-complete indicator indicating that the sterilization of the first filter has completed; and subsequent to the sterilization being completed, unlocking the first filter housing to provide access to the first filter.

In another aspect, the technology relates to a ventilation system that includes an inspiratory limb that carries breathing gases from a medical ventilator towards a patient; an expiratory limb that carries expiratory gases from the patient towards the medical ventilator; a dual-channel expiratory filtration system coupled to the expiratory limb and configured to receive unfiltered expiratory gases carried by the expiratory limb. The dual-channel expiratory filtration system includes an inlet that receives the unfiltered expiratory gases from the expiratory limb; a first channel, pneumatically coupled to the inlet, including a first filter that filters the unfiltered expiratory gases to form filtered expiratory gases; a second channel, pneumatically coupled to the inlet, including a second filter that filters the unfiltered expiratory gases to form filtered expiratory gases; a channel-selection mechanism configured to selectively control whether the unfiltered expiratory gases from the inlet flow through the first channel or the second channel; and an outlet, pneumatically coupled to the first channel and the second channel, that carries the filtered expiratory gases towards the ventilator.

In an example, the channel-selection mechanism comprises a first valve positioned in the first channel and a second valve positioned in the second channel. In another example, the channel-selection mechanism includes a rotatable valve that is: coupled to the inlet; selectively coupled to the first channel when the rotatable valve is in a first position; and selectively coupled to the second channel when the rotatable valve is in a second position. In another example, the channel-selection mechanism includes: a first component configured to compress tubing of the first channel to block gas flow through the first channel; and a second component configured to compress tubing of the second channel to block gas flow through the second channel. In still another example, the first component is a first elongate arm extending from a rotatable shaft, and the second component is a second elongate arm extending from the rotatable shaft. In yet another example, the first elongate arm includes a first magnet, and the second elongate arm includes a second magnet. In still yet another example, the channel-selection mechanism includes a cylindrical base, the cylindrical base including: an input port; a first-channel output port; and a second-channel output port; and a rotatable cylindrical cap, the rotatable cylindrical cap comprising: a cap-side input port; and a selection pathway.

In another example, the channel-selection mechanism includes an outer cylinder; an inlet coupled to the outer cylinder; a first-channel outlet port coupled to the outer cylinder; a second-channel outlet port coupled to the outer cylinder; a rotatable inner cylinder configured to rotate within the outer cylinder, the rotatable inner cylinder includes an inner cylinder inlet; and an inner cylinder selection pathway. In yet another example, the channel-selection mechanism includes a housing including an inlet, a first-channel outlet port, and second-channel outlet port; and a ball that is positionable to selectively block gas flow through either the first-channel outlet port or the second-channel outlet port. In still another example, the channel-selection mechanism is controlled by the ventilator. In a further example, the channel-selection mechanism is configured to be controlled manually by a clinician.

In another example, the system further includes a first check valve in a first post-filter segment of the first channel; and a second check valve in a second post-filter segment of the second channel. In still another example, the system further includes a sterilization system configured to sterilize the first filter prior to removal of the first filter.

In another aspect, the technology relates to a method for an example method for filtering expiratory gases while maintaining pressure in a breathing circuit. The method includes receive an expiratory gas flow through a window of a frame, positioned in breathing circuit, and a first filter positioned in the window; slidably receiving a second filter through a filter-receiving opening of the frame while maintaining pressure in the breathing circuit; and while the second filter is being received, receive the expiratory gas flow through the window of the frame, the first filter, and the second filter.

In an example, the second filter remains in contact with the first filter as the second filter is being received. In another example, receiving the second filter causes the first filter to be ejected through a filter-ejection opening of the frame.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts a diagram illustrating an example of a medical ventilator connected to a human patient.

FIG. 2 depicts an example dual-channel expiratory filtration system.

FIG. 3 depicts an example filter housing.

FIG. 4 depicts an example filter housing with a sterilization system.

FIG. 5 depicts another example dual-channel expiratory filtration system in a first state.

FIG. 6 depicts the example dual-channel expiratory filtration system of FIG. 5 in a second state.

FIG. 7 depicts an example channel-selection system.

FIG. 8 depicts an exploded perspective view of another example channel-selection system.

FIG. 9 depicts an exploded side view of the example channel-selection system of FIG. 8.

FIG. 10 depicts a perspective view of the example channel-selection system of FIG. 8.

FIG. 11A depicts a front view of the example channel-selection system of FIG. 8 when the channel-selection system is in a first state.

FIG. 11B depicts a front view of the example channel-selection system of FIG. 8 when the channel-selection system is in an intermediate state.

FIG. 12 depicts a perspective view of another example channel-selection system.

FIG. 13 depicts a top view of the example channel-selection system of FIG. 12.

FIG. 14 depicts an exploded perspective view of the example channel-selection system of FIG. 12.

FIG. 15 depicts a side view of a rotatable cylinder of the example channel-selection system of FIG. 12.

FIG. 16 depicts another example channel-selection system.

FIG. 17 depicts another example channel-selection system.

FIG. 18 depicts another example channel-selection system.

FIG. 19 depicts another example channel-selection system.

FIG. 20 depicts another example channel-selection system.

FIG. 21 depicts another example channel-selection system.

FIG. 22 depicts another example channel-selection system.

FIG. 23 depicts a variation of the example channel-selection system of FIG. 22.

FIG. 24 depicts another variation of the example channel-selection system of FIG. 22.

FIG. 25 depicts a perspective view of a replaceable filter system.

FIG. 26 depicts another perspective view of the replaceable filter system of FIG. 25.

FIG. 27 depicts a plurality of connected filter media.

FIG. 28 depicts an example method for selecting a filtration channel.

FIG. 29 depicts an example method for replacing an expiratory filter.

FIG. 30 depicts an example method for filtering expiratory gases while maintaining pressure in the breathing circuit.

FIG. 31 depicts an example method for filtering expiratory gases while maintaining pressure in the breathing circuit.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. In dual-limb ventilation systems, gases that are exhaled from the patient flow through tubing back to the ventilator.

Because the gases exhaled from the patient may include various substances, such as fluids, mucus, germs, bacteria, viruses, etc., exhaled gases are filtered prior to being received by the ventilator and being released back into the environment. Such filtration helps prevent the substances from interfering with ventilator functions or otherwise clogging passageways of the ventilator. The filtration of the gases also helps remove the germs, bacteria, and/or viruses that may be in the exhaled air. Thus, the filtered exhaled air can be exhausted from the ventilator back into the room without risk of introducing such pathogens into the environment.

The expiratory filters that perform the filtration have limited lifetimes and may become clogged or otherwise performance-degraded over time during ventilation. As a result, the filter needs to be replaced at regular intervals, and such replacement is often performed by a clinician. For patients that are being ventilated for long periods of time, the filter is replaced while the patient is still being ventilated. Removal of the dirty filter to insert the clean filter, however, breaks the breathing circuit and pressurization to the patient is lost. For instance, positive end-expiratory pressure (PEEP) is no longer able to be maintained. In addition, breaking of the circuit also causes air exhaled from the patient to enter the room or environment in an unfiltered state, which may introduce undesired pathogens into the air of the room.

Among other things, the present technology looks to alleviate the above problems with new expiratory filtration systems that allow for expiratory filters to be changed or replaced without having to break the circuit or interrupt ventilation of the patient. For example, the present technology includes a dual-channel expiratory filtration system that allows for gases to flow through one filter while another filter is replaced. In the dual-channel expiratory filtration system, two expiratory gas filters are provided in parallel channels at the end of the expiratory limb of the breathing circuit. Flow may then be toggled between one channel or the other to have the expiratory gases pass through one filter or the other. For instance, a first channel may be selected or toggled to have the expiratory gases flow through a first filter. While the gases are flowing through the first channel, gases are not flowing through the second channel. Thus, the filter in the second channel may be removed and replaced without having to break the circuit or interrupt ventilation of the patient. In addition, because gases are not flowing through the second channel, unfiltered expiratory gases are not introduced into the environment during replacement of the second filter. When replacement of the first filter is then needed, the flow of gases is toggled to the second channel, and the first filter may be replaced with the same process and benefits as discussed above. In some examples, the filter may also be sterilized prior to removal such that exposure to pathogens is further reduced.

The present technology may also or alternatively use a slidable filtration system that allows for replacement of filters without breaking the circuit. In the slidable filtration system, a frame is used that slidably receives filters. When one filter needs to be replaced, another filter is slid into the frame that causes the dirty filter to be pushed out of the frame. Both filters remain in contact with one another such that air continues to flow through at least one of the filters during the replacement process. With these concepts in mind, several examples of expiratory filtration methods and systems are discussed below.

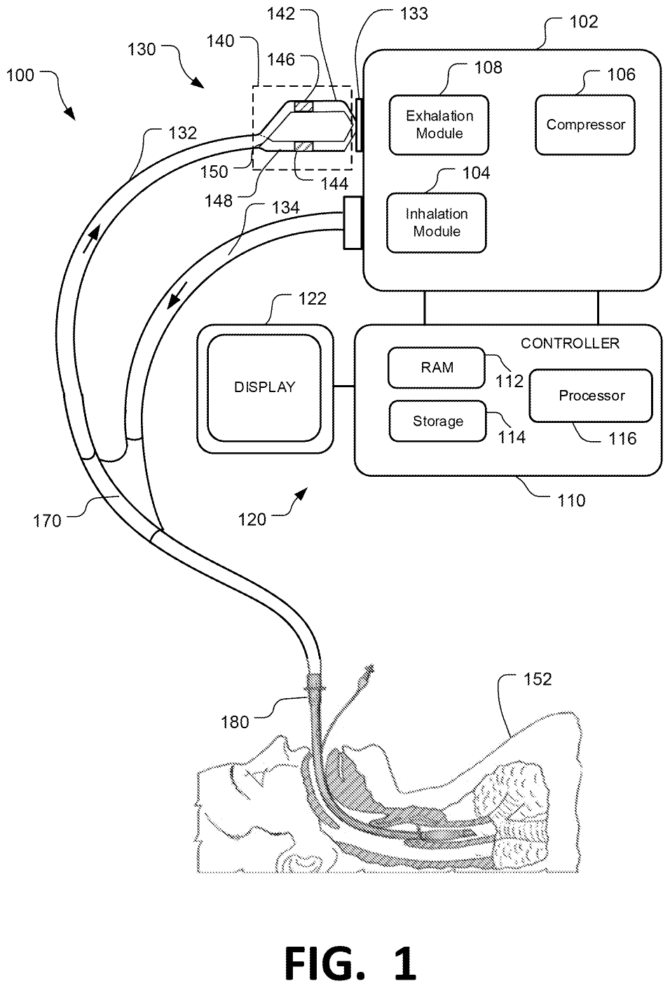

FIG. 1 is a diagram illustrating an example of a medical ventilator or ventilation system 100 connected to a human patient 152. The ventilator 100 may provide positive pressure ventilation to the patient 152. Ventilator 100 includes a pneumatic system 102 (also referred to as a pressure generating system 102) for circulating breathing gases to and from patient 152 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 152. In a two-limb example, a fitting, typically referred to as a “wye-fitting” 170, may be provided to couple a patient interface 180 to an inhalation or inspiratory limb 134 and an exhalation or expiratory limb 132 of the ventilation tubing system 130.

In the present technology, a dual-channel expiratory filtration system 140 is included at the end of the expiratory limb 132. The dual-channel expiratory filtration system 140 includes a first channel 142, with a first filter 146, and a second channel 144 with a second filter 148. The first channel 142 and the second channel 144 may be formed of tubing that may be the same or different from the remainder the tubing used to form the expiratory limb 132. For instance, each of the first channel 142 and the second channel 144 may have the same inner diameter as the tubing used for the expiratory limb 132. In some examples, the tubing of the first channel 142 and the second channel 144 may be formed of tubing that is more pliable (e.g., less rigid) than the expiratory limb 132 to allow for selective pinching or compressing of the tubing in the first channel 142 and the second channel 144. Such tubing may be peristaltic tubing, for example.

The dual-channel expiratory filtration system 140 also includes a channel-selection mechanism 150 to allow for selection or toggling between the first channel 142 and the second channel 144. For instance, the channel-selection mechanism 150 may be manipulated to cause gases to flow through the first channel 142 or the second channel 144. The channel-selection mechanism 150 may take a variety of forms, several of which are discussed below in more detail. The channel-selection mechanism 150 may be manually operated by a clinician and/or controlled by the ventilator 100 (e.g., by the controller 110).

The dual-channel expiratory filtration system 140 may be connected to or integrated into an expiratory port 133 of the ventilator 100. The expiratory port 133 may include a plurality of components and/or sensors, such as a flow sensor and/or a pressure sensor. Unfiltered gases that reach such sensors may interfere with the operation of the sensors. As such, the improved filtration benefits that result from the dual-channel expiratory filtration system 140 also increase the longevity and reliability of such sensors of the ventilator that interact with the expiratory gases.

Pneumatic system 102 may have a variety of configurations. In the present example, pneumatic system 102 includes an exhalation module 108 coupled with the expiratory limb 132 and an inhalation module 104 coupled with the inspiratory limb 134. Compressor 106 or other source(s) of pressurized gases (e.g., air, oxygen, and/or helium) is coupled with inhalation module 104 to provide a gas source for ventilatory support via inspiratory limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator).

Controller 110 is operatively coupled with pneumatic system 102, signal measurement and acquisition systems, and an operator interface 120 that may enable an operator to interact with the ventilator 100 (e.g., change ventilation settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, the computer-readable storage media may be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid-state memory technology, CD-ROM, DVD, or other optical storage, magnetic cassettes, magnetic tape, magnetic disk storage or other magnetic storage devices, or any other medium which can be used to store the desired information and which can be accessed by the computer. Communication between components of the ventilator system or between the ventilator system and other therapeutic equipment and/or remote monitoring systems may be conducted over a distributed network, via wired or wireless means.

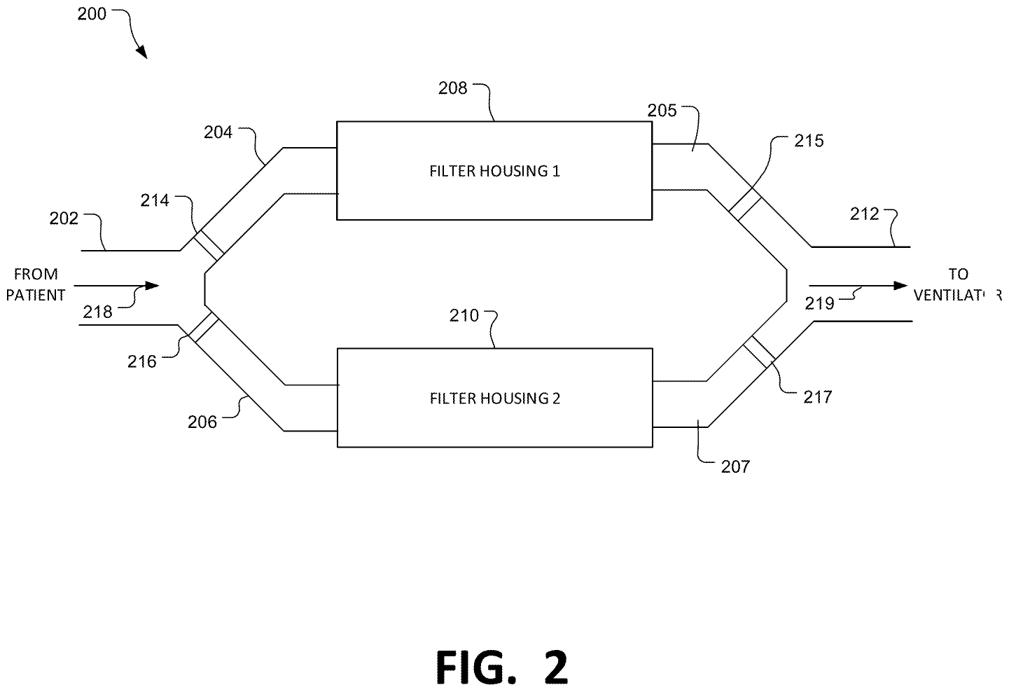

FIG. 2 depicts an example dual-channel expiratory filtration system 200. The dual-channel expiratory filtration system 200 includes an inlet 202 that connects to the expiratory limb of the patient circuit. The inlet 202 then splits into, or is coupled to, a first channel 204 and a second channel 206. The first channel 204 includes or is coupled to a first filter housing 208 such that gases flowing through the first channel 204 pass through the first filter housing 208. The second channel 206 includes or is coupled to a second filter housing 210 such that gases flowing through the second channel 206 pass through the second filter housing 210. The first channel 204 includes a first post-filter segment 205, and the second channel 206 includes a second post-filter segment 207. The first post-filter segment 205 and the second post-filter segment 207 join or combine at an outlet 212 of the dual-channel expiratory filtration system 200.

The first channel 204 includes a first selection valve 214, and the second channel includes a second selection valve 216. The first post-filter segment 205 includes a first check valve 215, and the second post-filter segment 207 includes a second check valve 217. The first check valve 215 and the second check valve 217 allow air to flow through the first channel 204 and the second channel 206, respectively, in a single direction (e.g., from the patient end to the ventilator end).

The first selection valve 214 and the second selection valve 216 operate as a channel selection mechanism or system. For instance, when the first selection valve 214 is open and the second selection valve 216 is closed, unfiltered expiratory gases 218 from the expiratory limb flow through the first channel 204 and the first filter housing 208 where the unfiltered expiratory gases are filtered to form filtered expiratory gases 219. The filtered expiratory gases 219 flow through the first check valve 215 and into the outlet 212. The filtered expiratory gases 219 then flow into the expiratory port of the ventilator. The second check valve 217 prevents the filtered expiratory gases 219 from flowing back into the second filter housing 210. As such, while the gases are flowing through the first channel 204 and the first filter housing 208, the second filter housing 210 can be opened or removed to replace the filter inside the second filter housing 210.

By controlling the first selection valve 214 and the second selection valve 216, gas flow may then be toggled to the second channel 206. For instance, by closing the first selection valve 214 and opening the second selection valve 216, the unfiltered expiratory gases 218 flow through the second channel 206 and the second filter housing 210. The filtered expiratory gases 219 then flow through the second check valve 217 and out the outlet 212. While the gases are flowing through the second channel 206, the first filter housing 208 may be removed or accessed to replace the filter therein. Selection mechanisms or components other than the first selection valve 214 and/or second selection valve 216 may be used to selectively control which channel the gases flow through, as discussed further herein. The channel-selection mechanisms may be controlled manually through physical interaction by the clinician and/or automatically controlled by the ventilator.

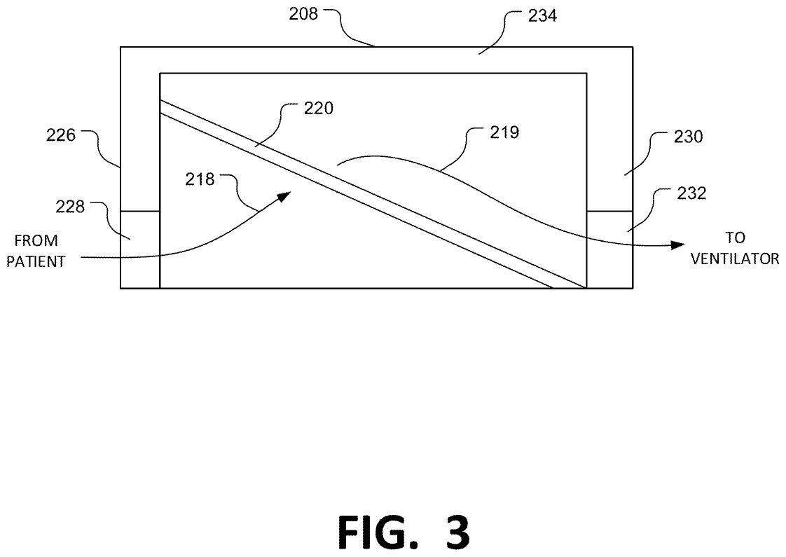

FIG. 3 depicts an example filter housing 208 with a filter media 220. The view shown in FIG. 3 is a cross-section of the filter housing 208 that shows at least a portion of the interior components of the filter housing 208, such as the filter media. The example filter housing 208 may be the first filter housing 208 shown in FIG. 2. The filter housing 208 includes an outer structure that may have a front side or patient side 226, a back side or ventilator side 230, and a top side 234. The patient side 226 includes an inlet port 228 to receive the unfiltered expiratory gases 218 into the first filter housing 208. The inlet port 228 may also receive a portion of the tubing that forms the respective channel, such as the first channel 204. When the unfiltered expiratory gases 218 flow into the first filter housing 208, the unfiltered expiratory gases 218 flow through the filter media 220 to form filtered expiratory gases 219. The filtered expiratory gases 219 exit through an outlet port 232 formed in the ventilator side 230 of the housing 208. The outlet port 232 may also receive a portion of the tubing of the post-filter segment respective channel, such as the first post-filter segment 205 of the first channel 204.

The inlet port 228 and/or the outlet port 232 may also include mating structures or components for coupling to the portions of tubing of the respective channels. For instance, the mating structures may include ridges, groves, threads, gaskets, or the like. The mating structures allow for a sealed coupling between the first filter housing 208 and the tubing of the respective channel such that gases do not exit or escape the channel or first filter housing 208.

In the example depicted, the filter or filter media 220 is positioned at an angle relative to the flow of gases coming into housing 208. Positioning the filter media 220 at such a non-orthogonal angle to the flow of gases helps improve the longevity of the filter media 220 by reducing localized particle accumulation associated with jetting. For instance, when the filter media 220 is positioned orthogonal to the stream of gases, the substances filtered from the gases accumulate in a small or narrow area of the filter media 220 (e.g., a small-radius spot) while the other portions of the filter media 220 are effectively unused. By angling the filter media 220, flow of the gases is diverted and dispersed in different directions such that the gases permeate the filter over a larger surface area of the filter media 220, which increases the longevity of the filter media 220 (e.g., the filter media 220 needs to be replaced less often). The angle of the filter media 220 relative to the flow of gases in some examples may be between 20-70 degrees or 30-60 degrees.

In other examples, the filter media 220 may be positioned in a manner where the filter media 220 is orthogonal to one or more sides of the housing 208, but the inlet port 228 and the outlet port 232 are not aligned with one another. For instance, the inlet port 228 may be located at a position on the patient side 226 that is different (e.g., higher) than the position of the outlet port 232 on the ventilator side 230, or vice versa. In such a configuration, the direction of the flow of gases is still not orthogonal to the surface of the filter media 220.

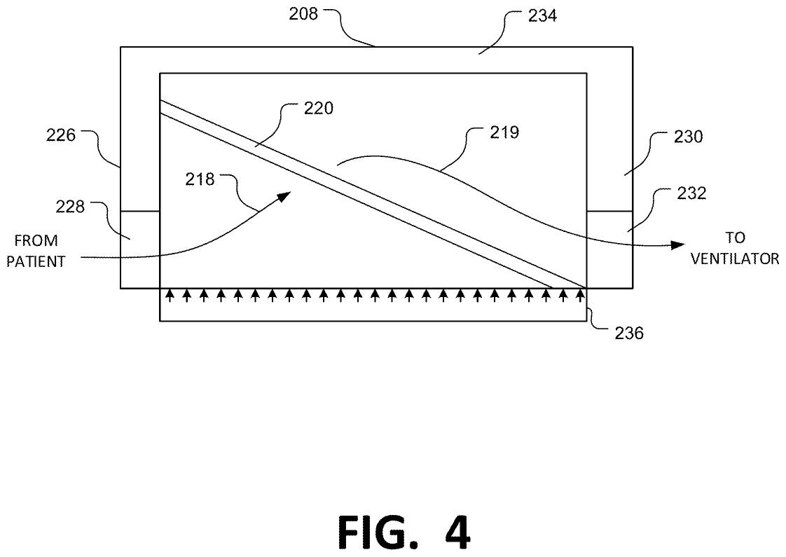

FIG. 4 depicts an example filter housing 208 with a sterilization system 236. The sterilization system 236 sterilizes the filter media 220 and, in some examples, may also at least partially sterilize the gases that are flowing through the housing 208. The sterilization system 236 may be an ultraviolet (UV) light-based sterilization system that includes a plurality of light sources (represented by example arrows in FIG. 4) that generate UV light (e.g., electromagnetic radiation in the UV spectrum). The UV light impinges the filter media 220 and sterilizes the filter media by killing or otherwise neutralizing pathogens trapped in the filter media 220. The UV light may also kill or neutralize pathogens in the gases flowing through the housing 208 or pathogens suspended in the gas/air of the housing 208 when gases are not flowing through the housing 208 (e.g., prior to a filter replacement).

The sterilization system 236 may be used to provide continuous sterilization, intermittent sterilization, and/or selective sterilization. For instance, the sterilization system 236 may be continuously activated to sterilize the filter media 220 and gases whenever ventilation is being provided to the patient. In other examples, the sterilization system 236 may be activated intermittently to sterilize the sterilization system 236 at different time periods, such as 30 seconds, 2 minutes, 5 minutes, 10 minutes, 1 hour, etc.

Additionally or alternatively, the sterilization system 236 may be selectively activated by a clinician input or in response to a triggering event. For instance, prior to replacement of the filter media 220, the filter media 220 may be sterilized by the sterilization system 236 to reduce risk to exposing the user to pathogens when the housing 208 is opened to remove the filter media 220. That sterilization process may be triggered/selected via input from the clinician prior to the replacement process. In other examples, when the gas flow is toggled from one channel to another channel, the sterilization process may be automatically triggered, such as by the ventilator, for the filter that is in the channel where no gases are flowing.

Activating the sterilization system 236 either continuously or intermittently during ventilation also provides benefits in addition to the sterilization of the filter media 220 and the gases. The sterilization system 236 generally provides heat in addition the UV light. The generated heat increases the temperature of the surrounding components, such as the housing 208 and tubing of the respective channels, along with the expiratory gases flowing therethrough. Increasing the temperature of the gases helps prevent rainout of moisture in the breathing gases (e.g., condensation from the temperature of the gases dropping below the dew point). Rainout may interfere with the sensors or other elements of the ventilator in the expiratory port as well as degrade the performance of the filter media 220. Because the housing 208 is located at the ventilator-end of the patient circuit (e.g., away from the patient), temperatures of the gases can be substantially increased without creating potential for injury or impact on the patient.

Additionally, the housing 208 may be designed such that any rainout that occurs in the housing 208 flows in a specific direction where the water may be trapped. For example, a bottom or lower surface (with respect to gravity), may be sloped such that any liquid flows in an intended direction. Such a watershed configuration helps prevent condensed liquid from flowing back into the patient circuit, particularly in the direction of the patient.

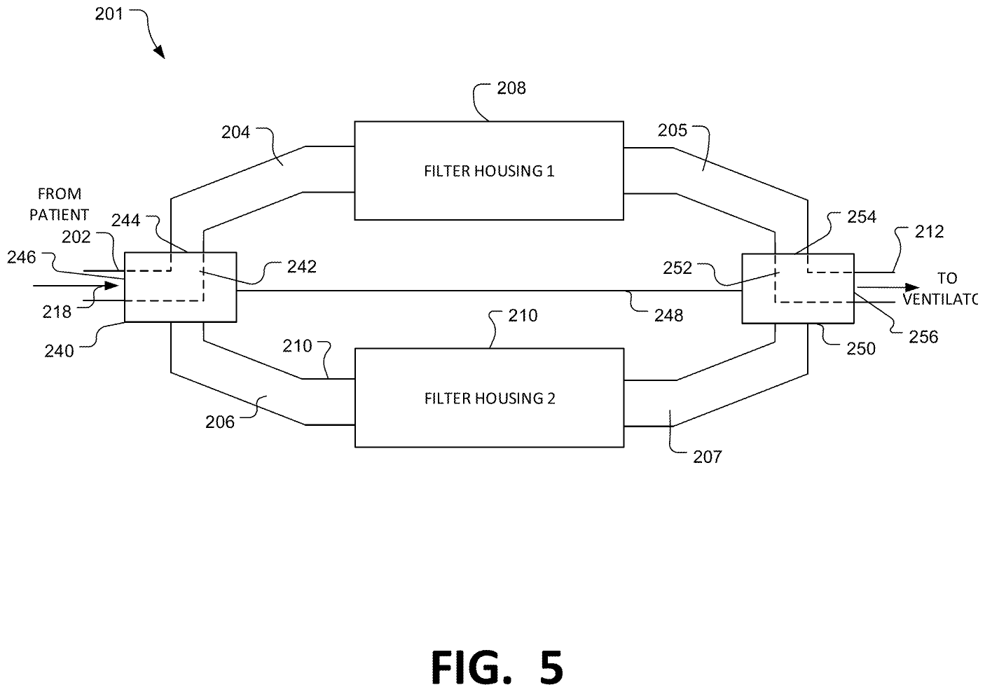

FIG. 5 depicts another example dual-channel expiratory filtration system 201 in a first state. The dual-channel expiratory filtration system 201 depicted in FIG. 5 uses a different channel selection mechanism than the channel-selection mechanism of the dual-channel expiratory filtration system 200 depicted in FIG. 2 and discussed above. The dual-channel expiratory filtration system 200 in FIG. 2 used a separate valve for each channel, whereas the dual-channel expiratory filtration system 201 in FIG. 5 uses two rotatable valves 240, 250. More specifically, the dual-channel expiratory filtration system 201 includes a first rotatable valve 240 located on a patient side of the filter housings 208, 210, and a second rotatable valve 250 located on a ventilator side of the filter housings 208, 210.

The first rotatable valve 240 includes an internal pathway 242 through which gases flow. The first rotatable valve 240 includes input port 246 at a patient end of the pathway 242 and an output port 244 at a ventilator end of the pathway 242. The second rotatable valve 250 includes a pathway 252 through which gases flow. The second rotatable valve 250 also includes an input port 254 at a patient end of the pathway 252 and an output port at a ventilator end of the pathway 252.

The input port 246 of the first rotatable valve 240 is coupled to the inlet 202 of the example dual-channel expiratory filtration system 201. The input port 246 may remain permanently coupled to the outlet 212 as the first rotatable valve 240 rotates. The output port 244, however, selectively couples with the first channel 204 or the second channel 206 depending on the rotation state of the first rotatable valve 240. In the first state depicted in FIG. 5, the output port 244 is coupled to the first channel 204. Accordingly, in the first state, the inlet 202 is pneumatically coupled to the first channel 204 via the internal pathway 242 and the unfiltered expiratory gases 218 flow through the first channel 204.

The output port 256 of the second rotatable valve 250 is coupled to the outlet 212 of the dual-channel expiratory filtration system 201. The output port 256 may remain permanently coupled to the outlet 212 as the second rotatable valve 250 rotates. The input port 254, however, selectively couples with the first post-filter segment 205 of the first channel 204 or the second post-filter segment 207 of the second channel 206. Accordingly, in the first state, the outlet 212 is pneumatically coupled to the first channel 204 via the internal pathway 252 and filtered expiratory gases 219 flow from the housing 208, through the first post-filter segment 205 and pathway 252, and out of the outlet 212 to the ventilator.

The first rotatable valve 240 and the second rotatable valve 250 may be connected via a rod 248 or other similar connection structure. Connecting the first rotatable valve 240 and the second rotatable valve 250 via the rod 248 helps maintain the first rotatable valve 240 and the second rotatable valve 250 in the same state. For instance, when connected by the rod 248, rotation of the second rotatable valve 250 causes rotation of the first rotatable valve 240, and vice versa. Rotation of the first rotatable valve 240 and the second rotatable valve 250 may also be accomplished by rotating the rod 248.

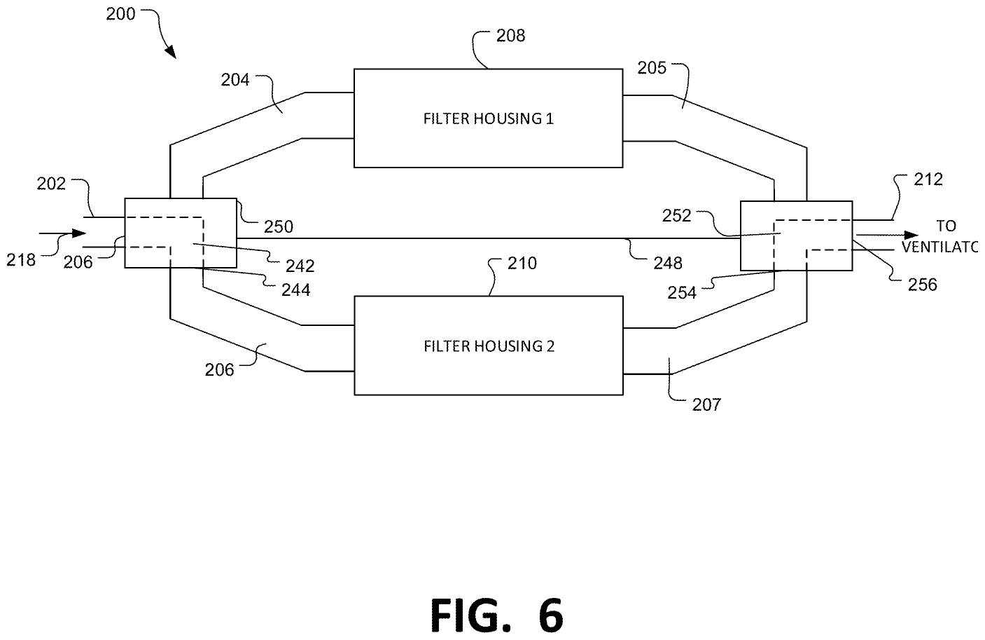

FIG. 6 depicts the example dual-channel expiratory filtration system 201 of FIG. 5 in a second state. In the second state, the first rotatable valve 240 and the second rotatable valve 250 have been rotated such that the second channel 206 has been selected. More specifically, the first rotatable valve 240 has been rotated such that the output port 244 is coupled to the second channel 206 rather than the first channel 204. Similarly, the second rotatable valve 250 has been rotated such that the input port 254 is coupled to the second post-filter segment 207 rather than the first post-filter segment 205. Thus, in the second state, the unfiltered expiratory gases 218 flow through the inlet 202, the internal pathway 242, and the second channel 206. The unfiltered expiratory gases 218 are filtered by the filter in the filter housing 210 to form filtered expiratory gases 219 that flow through the second post-filter segment 207, the pathway 252, and the outlet 212 where they are received by the ventilator. While the gases are flowing through the second channel 206 rather than the first channel 204, the filter in the housing 208 may be replaced without breaking the circuit or interrupting ventilation of the patient.

Rotation and/or control of the first rotatable valve 240, the second rotatable valve 250, and/or the rod 248 may be performed manually or automatically by the ventilator. For example, the ventilator may control a motor attached to one of the first rotatable valve 240, second rotatable valve 250, and/or the rod 248 to position the system 201 in either the first state or the second state.

In other variations of the dual-channel expiratory filtration system 201, the second rotatable valve 250 may be removed, and the check valves 215, 217 may be used instead. For example, the first rotatable valve 240 may be used for selectivity of the first channel 204 or the second channel 206, and the check valves 215, 217 prevent back flow into the unselected channel.

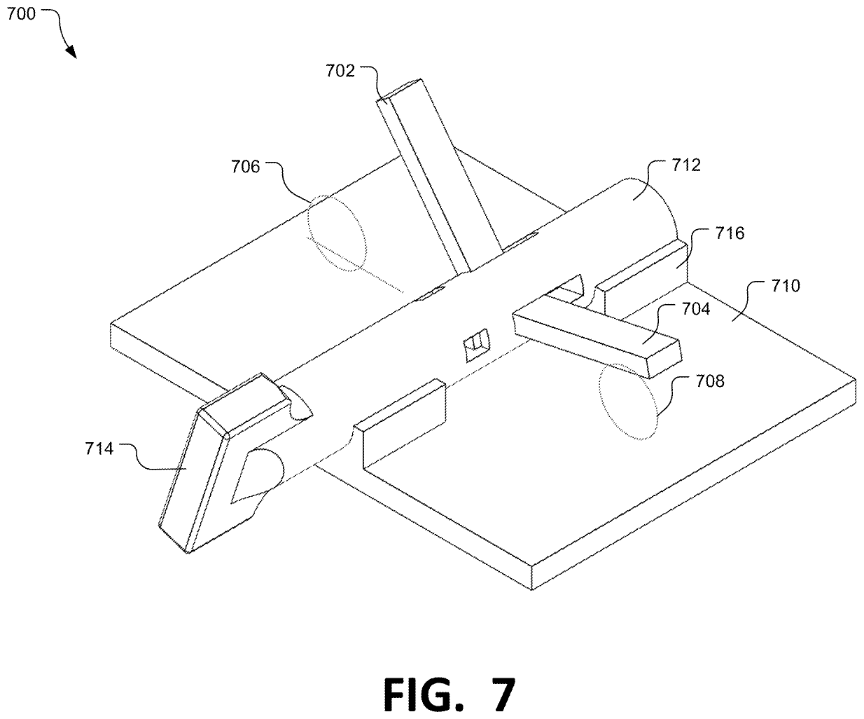

Other channel-selection mechanisms in addition to the valve systems discussed above are also possible. FIGS. 7-23 depict some of the different options available as a channel-selection mechanism for use with the example dual-channel expiratory filtration systems of the present technology.

FIG. 7 depicts one example channel-selection system 700. The channel-selection system includes a first elongate arm 702 and a second elongate arm 704 that are coupled to rotatable shaft 712 that includes a handle 714. The rotatable shaft 712 is positioned in a cradle 716 of a base 710 of the channel-selection system 700. The first arm 702 is configured to compress or pinch the tubing of a first channel represented by first channel indicator 706. The second arm 704 is configured to compress or pinch the tubing a second channel represented by the second channel indicator 708.

When the handle 714 is rotated in a first direction, rotation of the shaft 712 causes the first arm 702 to rotate downward and compress the tubing of the first channel such that no gases may flow through the first channel. Concurrently with the first arm 702 moving downwards towards the base, the rotation also cause the second arm 704 to raise away from the base. Thus, gases may flow through the second channel while the first channel is blocked by the compression caused by the first arm 702.

Conversely, when the handle 714 is rotated in a second direction (e.g., a direction opposite the first direction), rotation of the shaft 712 causes the second arm 704 to rotate downward and compress the tubing of the second channel such that no gases may flow through the second channel. Concurrently with the second arm 704 moving downwards towards the base 710, the first arm 702 raises away from the base 710. Thus, gases may flow through the first channel while the second channel is blocked by the compression caused by the second arm 704.

When the second arm 704 is compressing the tubing of the second channel and gases are flowing through the first channel, the channel-selection system 700 may be considered to be in the first state. When the first arm is compressing the tubing of the first channel and gases are flowing through the second channel, the channel-selection system 700 may be considered to be in the second state.

The channel-selection system 700 may also include mechanisms to hold the channel-selection system 700 in either the first state or the second state. For instance, the first arm 702 and/or the second arm 704 may each include a magnet, and the base 710 may be formed of a magnetic material such that when one of the arms 702, 704 is compressing the tubing against the base 710, the arm is held in place by the magnetic force between the magnet and the magnetic base 710. In other examples, pins, detents, or other types of locking mechanisms may used to hold the channel-selection system 700 in either the first state or the second state.

One benefit of the channel-selection system 700 over some of the other channel selection mechanisms/systems discussed herein is that the channel-selection system 700 itself is not directly exposed to the gases flowing through either of the channels. For instance, the arms 702, 704 compress the outside of the tubing, and thus the arms do not contact the gases flowing through the tubing. As a result, the channel-selection system 700 does not need to be disinfected, or can be less thoroughly disinfected, when the channel-selection system 700 is used with a new patient.

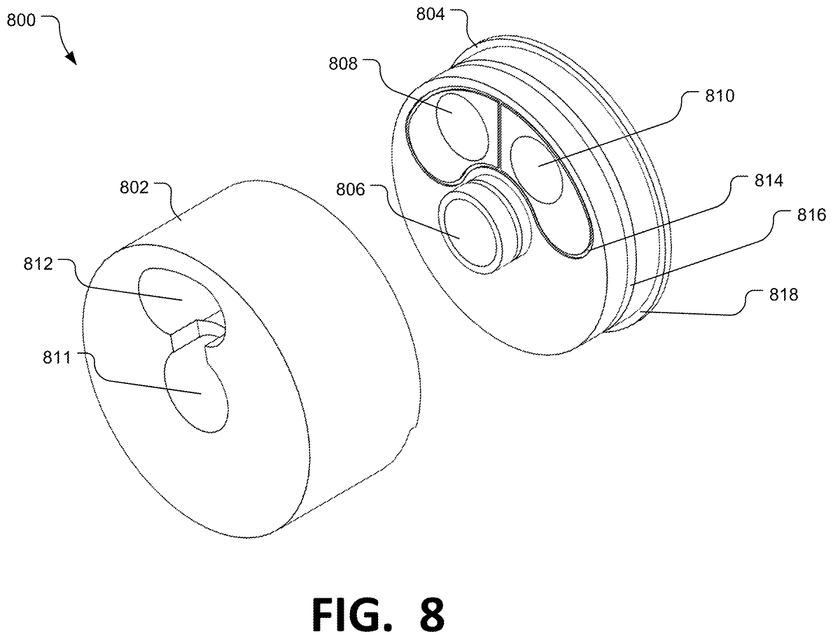

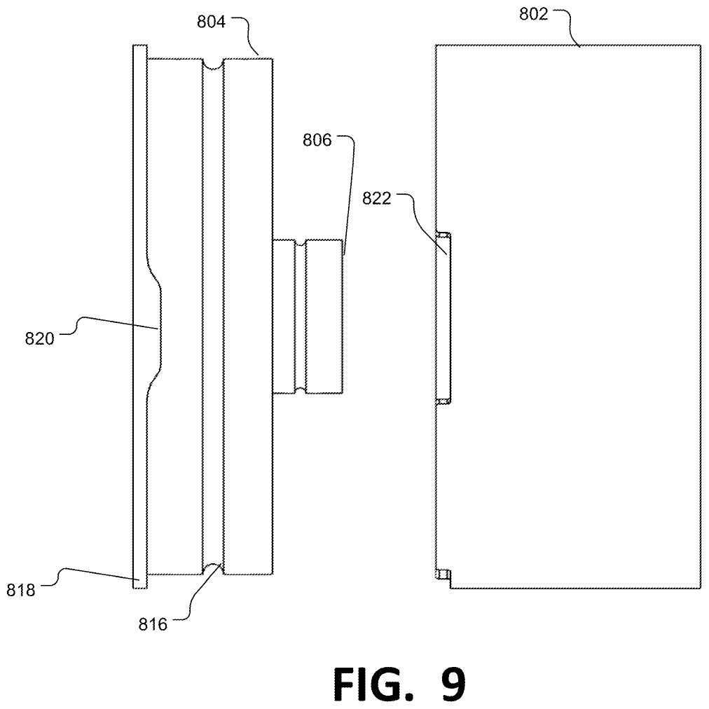

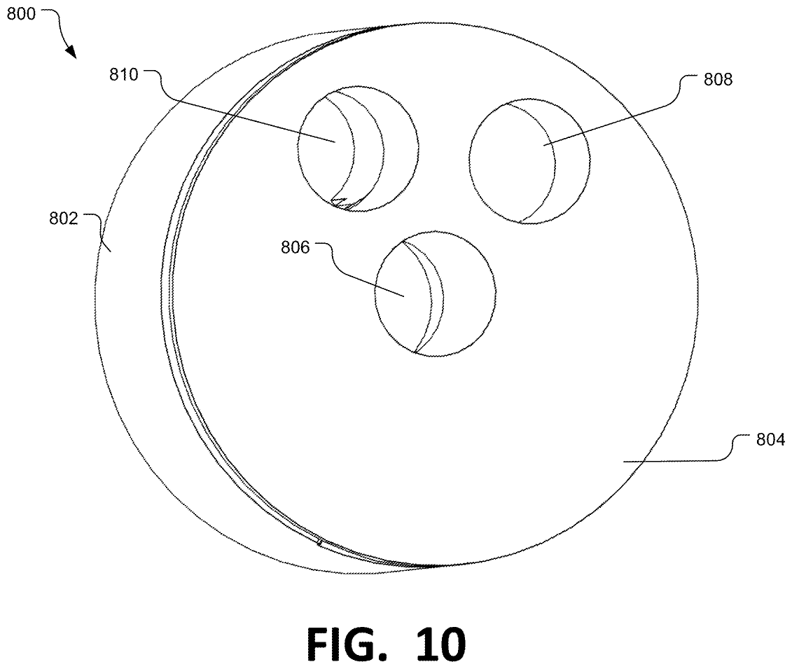

FIG. 8 depicts an exploded perspective view of another example channel-selection system 800. FIG. 9 depicts an exploded side view of the example channel-selection system 800 of FIG. 8. FIG. 10 depicts a perspective view of a rear side the example channel-selection system 800 of FIG. 8. FIGS. 8-10 are discussed concurrently.

The channel-selection system 800 includes a cylindrical base 804 and a rotatable cylindrical cap 802. The cylindrical base 804 includes an input port 806, a first-channel outlet port 808, and a second-channel outlet port 810. The input port 806 couples to an inlet of an expiratory limb of the patient circuit. The first-channel outlet port 808 couples to tubing of a first channel of a dual-channel expiratory filtration system and the second-channel outlet port 810 couples to tubing of a second channel of the dual-channel expiratory filtration system. Each of the ports remains coupled to the respective tubing during operation and selection of the respective channels.

Selection of one channel versus another channel is controlled through rotation of the rotatable cylindrical cap 802. Gases flowing through the input port 806 flow through the cap-side input port 811 and through the selection pathway 812 via a notch between the cap-side input port 811 and the selection pathway 812. The selection pathway 812 is an opening that, in some examples such as the one depicted, has a width that is greater than either one of the first-channel outlet port 808 or the second-channel outlet port 810. In a first state, the selection pathway 812 of the rotatable cylindrical cap 802 is aligned with the first-channel outlet port 808, which causes gases to flow through the input port 806 and out the first-channel outlet port 808 without any gases flowing through the second-channel outlet port 810. In a second state, the selection pathway 812 of the rotatable cylindrical cap 802 is aligned with the second-channel outlet port 810, which causes gases to flow through the input port 806 and out the second-channel outlet port 810 without any gases flowing through the first-channel outlet port 808.

When the rotatable cylindrical cap 802 is coupled to the cylindrical base 804, a gasket may be positioned in the groove 816 to seal the example channel-selection system 800 such that gas escapes only via the respective selected outlet. In addition, the gasket 814 surrounds an outer perimeter of the first-channel outlet port 808 and the second-channel outlet port 810 to further prevent gas leakage. A portion of the gasket 814 may be shaped so as to match a perimeter shape of the selection pathway 812 of the rotatable cylindrical cap 802.

The cylindrical base 804 may also include a flange 818 that protrudes radially outward from the perimeter of the cylindrical base 804. The flange 818 provides a shelf or surface that is in contact with the rotatable cylindrical cap 802 when the rotatable cylindrical cap 802 and the cylindrical base 804 are coupled together. The cylindrical base 804 may also include tabs 820 that protrude from the flange 818 in the direction of the rotatable cylindrical cap 802. In such examples, the rotatable cylindrical cap 802 may have matching notches 822 that align with the tabs 820. When in the first state, one notch 822 of the rotatable cylindrical cap 802 aligns with a tab 820 of the cylindrical base 804. In the second state, another notch 822 aligns with the tab 820 of the cylindrical base 804. When the tab 820 is aligned with a notch 822, additional resistance to rotation of the rotatable cylindrical cap 802 is generated, which helps hold the example channel-selection system 800 in a particular state. To change the state of the example channel-selection system 800, the rotatable cylindrical cap 802 may need to be pulled apart slightly from the cylindrical base 804 such that the tabs 820 and the notches 822 do not interfere with one another during rotation. The amount that the cap 802 and base 804 need to be pulled apart is based on the height or amount of protrusion of the tabs 820.

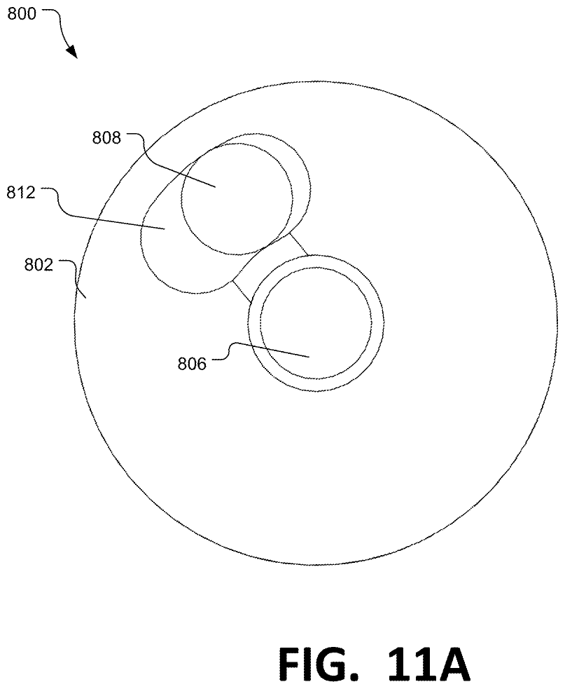

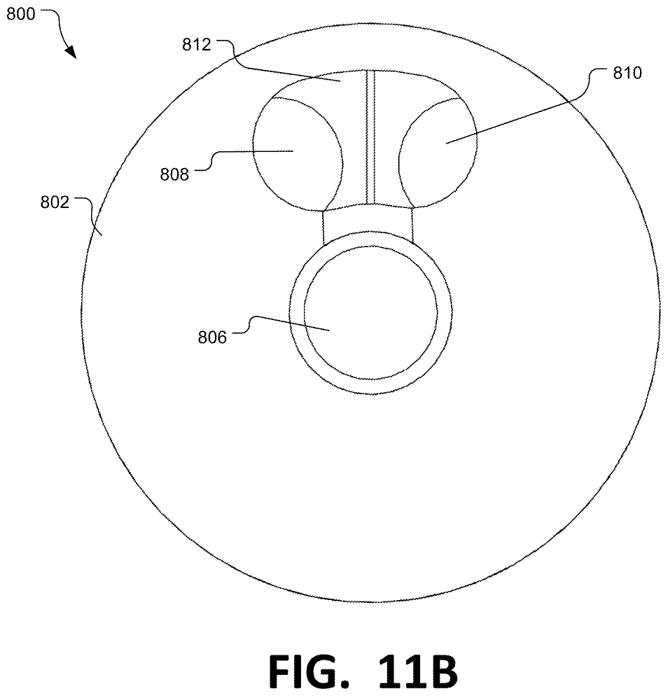

FIG. 11A depicts a front view of the example channel-selection system 800 when the channel-selection system 800 is in the first state. In the first state, the selection pathway 812 is positioned such that air may flow through the input port 806 and out the first-channel outlet port 808, but not through the second-channel outlet port 810.

FIG. 11B depicts a front view of the example channel-selection system of FIG. 8 when the channel-selection system is in an intermediate state when the rotatable cylindrical cap 802 is being rotated relative to the cylindrical base 804. Due to the selection pathway 812 being larger than the first-channel outlet port 808 or the second-channel outlet port 810, a portion of the first-channel outlet port 808 and the second-channel outlet port 810 may be aligned with the selection pathway 812 during rotation of the rotatable cylindrical cap 802. For instance, as shown in FIG. 11B, when rotation of the cap 802 is halfway between the first state and the second state, roughly half of the first-channel outlet port 808 and half of the second-channel outlet port 810 is aligned with the selection pathway 812. In such an intermediate state, gas flows from the input port 806 and out both of the first-channel outlet port 808 and the second-channel outlet port 810. In some examples, the same total area of openings of the outlet ports 808, 810 are exposed through the selection pathway 812 for every intermediate state during rotation.

Having gases flow through both outlets during the intermediate states (e.g., during rotation of the rotatable cylindrical cap 802) allows for the full flow of gases to flow through the channel-selection system 800 even during rotation. Thus, changing from one state to another state has minimal impact on the ventilation that is being provided to the patient as well as any resistance to exhalation.

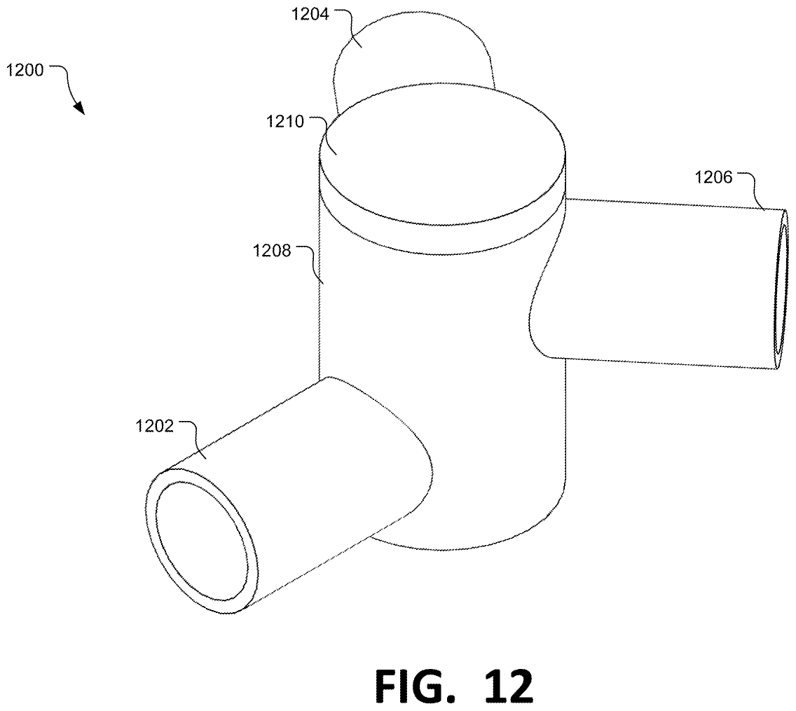

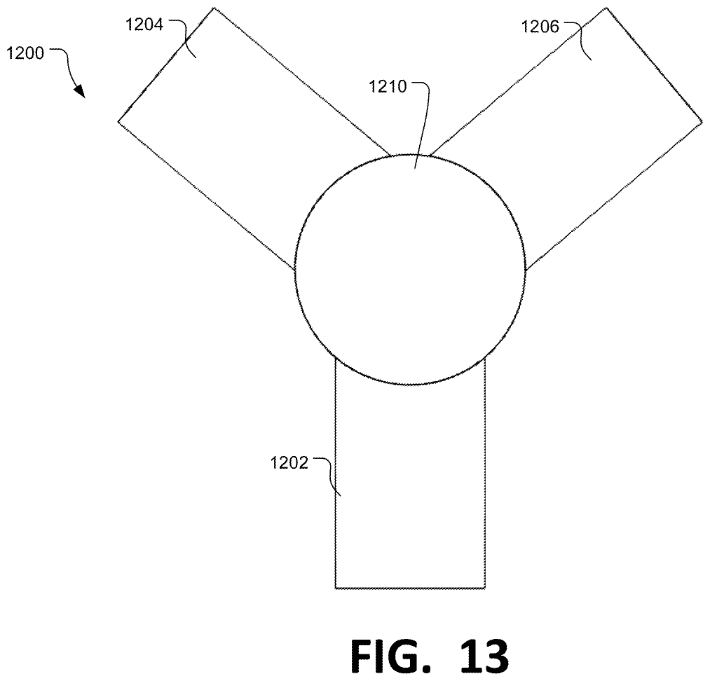

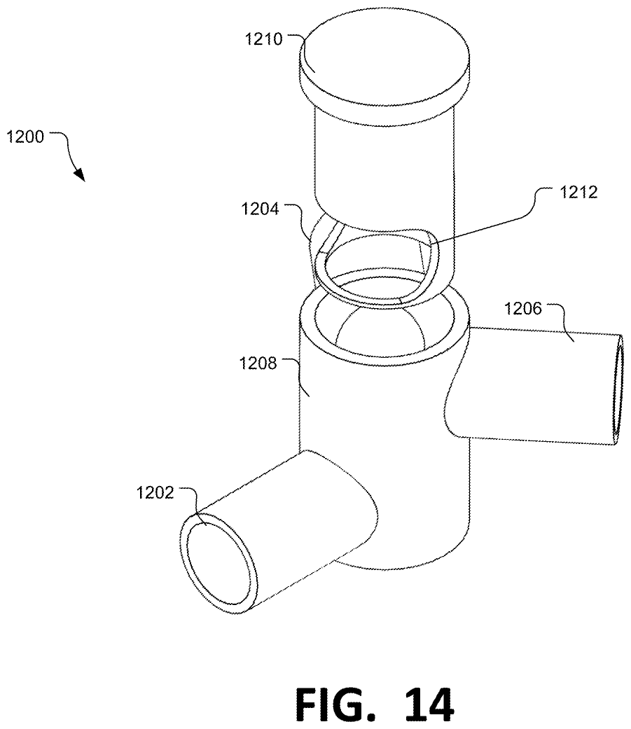

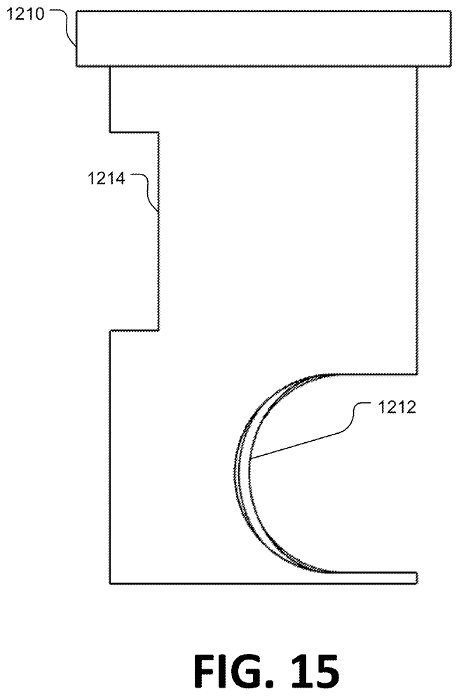

FIG. 12 depicts a perspective view of another example channel-selection system 1200. FIG. 13 depicts a top view of the example channel-selection system 1200. FIG. 14 depicts an exploded perspective view of the example channel-selection system 1200. FIG. 15 depicts a side view of a rotatable cylinder 1210 of the example channel-selection system 1200. FIGS. 12-15 are discussed concurrently.

The example channel-selection system 1200 includes an outer cylinder 1208 with an inlet 1202, a first-channel outlet port 1204, and a second-channel outlet port 1206. An inner rotatable cylinder 1210 resides inside the outer cylinder 1208. The rotatable cylinder 1210 includes an inner-cylinder inlet 1212 and an inner-cylinder selection pathway 1214.

The inlet 1202 is positioned lower on the outer cylinder 1208 than the first-channel outlet port 1204 and the second-channel outlet port 1206, which are posited at substantially the same height on the outer cylinder 1208. The inner-cylinder inlet 1212 is positioned at a height that aligns with the inlet 1202, and the inner-cylinder selection pathway 1214 is positioned at a height that aligns with the height of the first-channel outlet port 1204 and the second-channel outlet port 1206.

Rotation of the rotatable cylinder 1210 causes the example channel-selection system 1200 to change from a first state to a second state. For instance, in a first state, the rotatable cylinder 1210 is rotated to a position where the inner-cylinder selection pathway 1214 is aligned with the first-channel outlet port 1204. In the first state, expiratory gases flow into the inlet 1202 and out the first-channel outlet port 1204, which may be coupled to tubing of a first channel of a dual-channel expiratory filtration system 200. For example, the expiratory gases flow through the inlet 1202, the inner-cylinder inlet 1212, up the inner cavity of the rotatable cylinder 1210, through the inner-cylinder selection pathway 1214, and out the first-channel outlet port 1204.

In a second state, the rotatable cylinder 1210 is rotated such that the inner-cylinder selection pathway 1214 aligns with the second-channel outlet port 1206 and not the first-channel outlet port 1204. The inner-cylinder inlet 1212, however, remains aligned with the inlet 1202 because the inner-cylinder inlet 1212 is substantially larger than the inner-cylinder selection pathway 1214. For example, the inner-cylinder inlet 1212 has a width such that the inner-cylinder inlet 1212 remains aligned with the inlet 1202 as the rotatable cylinder 1210 rotates from the first state to the second state. In the second state, expiratory gases flow into the inlet 1202 and out the second-channel outlet port 1206.

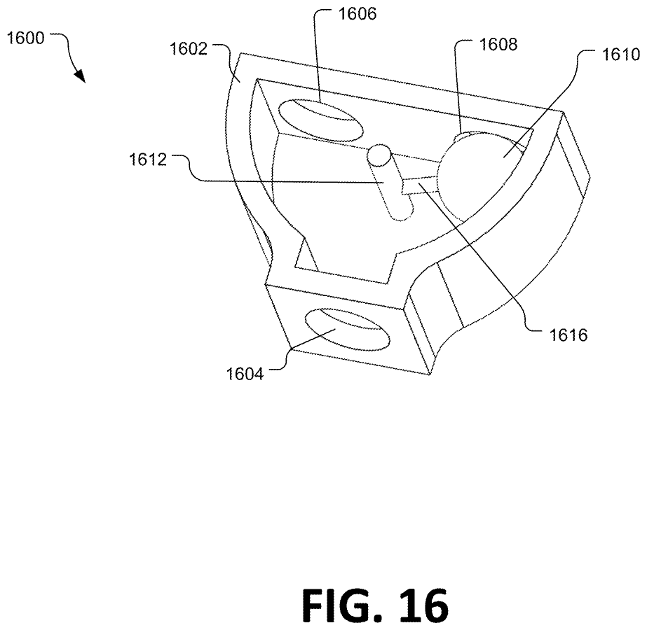

FIG. 16 depicts another example channel-selection system 1600. The example channel-selection system 1600 includes a housing 1602 that includes an inlet 1604, a first-channel outlet port 1606 and a second-channel outlet port 1608. The inlet 1604, a first-channel outlet port 1606 and a second-channel outlet port 1608 may be formed as through holes in the housing. The inlet 1604 is coupled to an expiratory limb of the patient circuit, the first-channel outlet port 1606 is coupled to tubing of a first channel, and the second-channel outlet port 1608 is coupled to tubing for the second channel.

The channel-selection system 1600 also includes a ball 1610 that may be selectively positioned in either the second-channel outlet port 1608 or the first-channel outlet port 1606. When the ball 1610 is positioned within a port, gas flow through that port is blocked. The ball 1610 may be attached to a rod 1616 that connects the ball 1610 to a shaft 1612. The ball 1610 and the rod 1616 may then be rotated about the shaft 1612 to selectively position the ball 1610 in either the first-channel outlet port 1606 or the second-channel outlet port 1608.

When the channel-selection system 1600 is in the first state, as depicted in FIG. 16, the ball 1610 is positioned in, and blocks, the second-channel outlet port 1608. Accordingly, gases flowing into the inlet 1604 flow out the first-channel outlet port 1606 but not the second-channel outlet port 1608. Because a positive flow and pressure of the expiratory gases is often maintained during ventilation, the ball 1610 is held in place by the pressures of the gases. The ball 1610 may also be held in place by frictional forces between the ball 1610 and the second-channel outlet port 1608.

The channel-selection system 1600 may be changed to the second state by moving the ball 1610 to the first-channel outlet port 1606. When in the second state, gases flow into the inlet 1604 and flow out the second-channel outlet port 1608 but not the first-channel outlet port 1606.

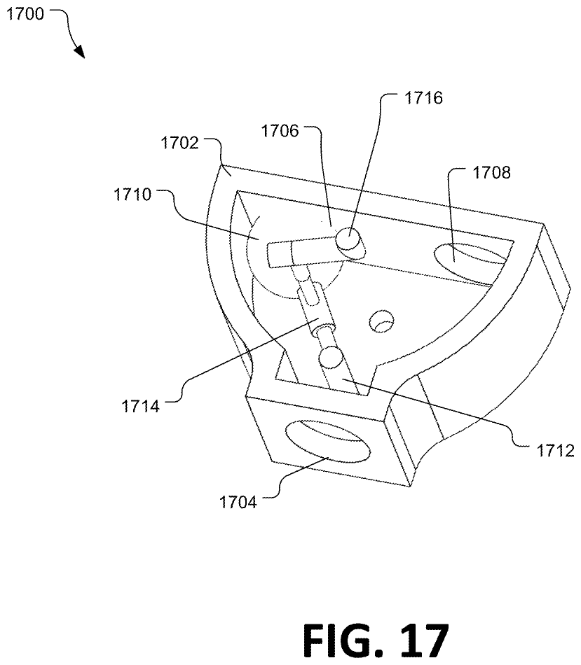

FIG. 17 depicts another example channel-selection system 1700. The channel-selection system 1700 is substantially similar to the channel-selection system 1600 described above with respect to FIG. 16. For instance, the channel-selection system 1700 uses a single repositionable ball 1710 to select either a first-channel outlet port 1706 or a second-channel outlet port 1708. Thus, air flowing through an inlet 1704 of the housing 1702 can be selectively controlled to either flow through the first-channel outlet port 1706 or the second-channel outlet port 1708.

The channel-selection system 1700, however, also uses a spring force to hold the ball 1710 in either the first-channel outlet port 1706 or the second-channel outlet port 1708. The ball 1710 still rotates around a shaft 1712. The rod 1714 that connects the ball 1710 to the shaft 1712 is spring-loaded. The spring-loaded rod 1714 may be separately connected to another shaft 1716 to provide additional support for the spring-loading forces.

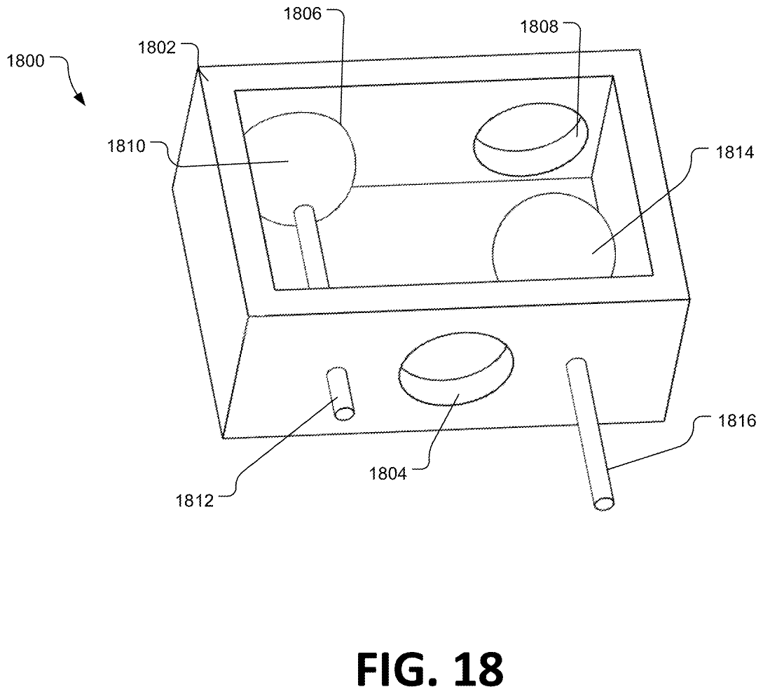

FIG. 18 depicts another example channel-selection system 1800. The example channel-selection system 1800 includes housing 1802. The housing 1802 includes an inlet 1804, a first-channel outlet port 1806, and a second-channel outlet port 1808. The channel-selection system 1800 also includes a first ball 1810 attached to a first rod 1812 and a second ball 1814 attached to a second rod 1816.

The positioning of the first ball 1810 and the second ball 1814 allows for selection of a state of the channel-selection system 1800. For instance, in a first state, the second ball 1814 is positioned in the second-channel outlet port 1808 and blocks gas flow through the second-channel outlet port 1808. In the first state, the first ball 1810 is positioned within the housing 1802 but not in the first-channel outlet port 1806. As such, in the first state, gas flows in through the inlet 1804 and out the first-channel outlet port 1806.

In a second state, the first ball 1810 is positioned in the first-channel outlet port 1806 and blocks gas flow through the first-channel outlet port 1806. In the second state, the second ball 1814 is not positioned in the second-channel outlet port 1808. Accordingly, gas may flow through the second-channel outlet port 1808. In the second state, gas flows in through the inlet 1804 and out the second-channel outlet port 1808.

Changing the channel-selection system 1800 from the first state to the second state (and vice versa) may be controlled through manipulation of the rods 1812, 1816. The rods 1812, 1816 may protrude through the housing 1802 such that the rods 1812, 1816 may be accessed from outside of the housing 1802. Pushing or pulling on the first rod 1812 causes a translational, linear movement of the first ball 1810 within the housing 1802. For instance, pushing on the first rod 1812 may position the first ball 1810 within the first-channel outlet port 1806, and pulling the first rod 1812 may remove the first ball 1810 from the first-channel outlet port 1806. Similarly, pushing or pulling of the second rod 1816 causes a translational, linear movement of the second ball 1814.

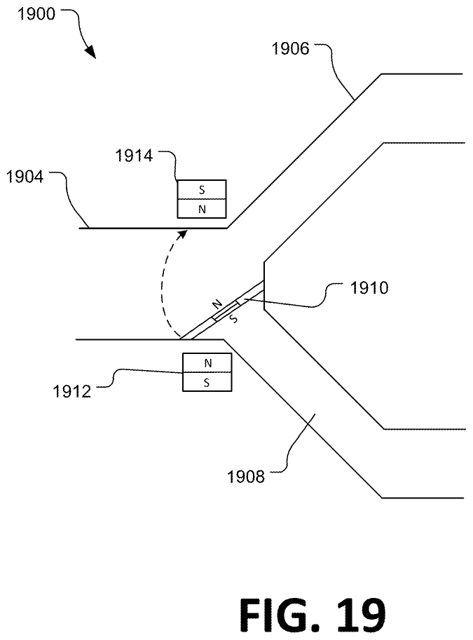

FIG. 19 depicts another example channel-selection system 1900. The channel-selection system 1900 includes an inlet 1904, a first channel 1906, and a second channel 1908. The system 1900 also includes a gate or flap 1910 that may be magnetically controlled through a first magnet 1912 and a second magnet 1914. The first magnet 1912 may be positioned on one side of the tubing that forms the inlet 1904 and the second magnet 1914 may be positioned on another side of the tubing, which may be a substantially opposite side.

The position of the flap 1910 controls the state of the channel-selection system 1900. For instance, in a first state (as shown in FIG. 19), the flap 1910 is positioned to block gas flow into the second channel 1908. Thus, in the first state, gas flows into the inlet 1904 and through the first channel 1906. In a second state, the flap 1910 moves (e.g., pivots) to block the flow of gas through the first channel 1906. Thus, in the second state, gas flows into the inlet 1904 and out of the second channel 1908.

The flap 1910 includes a magnet with a first pole (e.g., south pole) facing towards the first magnet 1912 and the second pole (e.g., north pole) facing away from the first magnet 1912. The first magnet 1912 may then be rotated to either attract or repel the magnet of the flap 1910. For instance, when a north pole of the first magnet 1912 faces the south pole of the magnet in the flap 1910, the flap 1910 is attracted to, and moves towards, the first magnet 1912. Conversely, when the first magnet 1912 is rotated such that the south pole of the first magnet 1912 faces the south pole of the flap 1910, the flap 1910 is repelled by, and moves away from, the first magnet 1912. In some examples, the magnetic forces between the flap 1910 and the first magnet 1912 are sufficient to move the flap 1910 and hold the flap 1910 in a particular position (e.g., the first state or the second state). In other examples, a second magnet 1914 may be used to increase the magnetic forces.

The second magnet 1914 operates similarly to the first magnet. For instance, when a north pole of the second magnet 1914 faces the north pole of the magnet in the flap 1910, the flap 1910 is repelled by the second magnet 1914. When the south pole of the second magnet faces the north pole of the magnet in the flap 1910, the flap is attracted to the second magnet 1914. Accordingly, when the first 1912 is positioned to attract the flap 1910, the second magnet 1914 is positioned to repel the flap 1910. When the first magnet 1912 is positioned to repel the flap 1910, the second magnet 1914 is positioned to attract the flap 1910.

While not depicted, the first magnet 1912 and the second magnet 1914 may be physically connected to one another such that rotation one magnet causes rotation of the second magnet. As another example, rather than each magnet being individually rotatable, the magnets may be rotated around the tubing that forms the inlet 1904. For instance, to change states of the channel-selection system 1900, the position of the first magnet 1912 may be swapped with the position of the second magnet 1914.

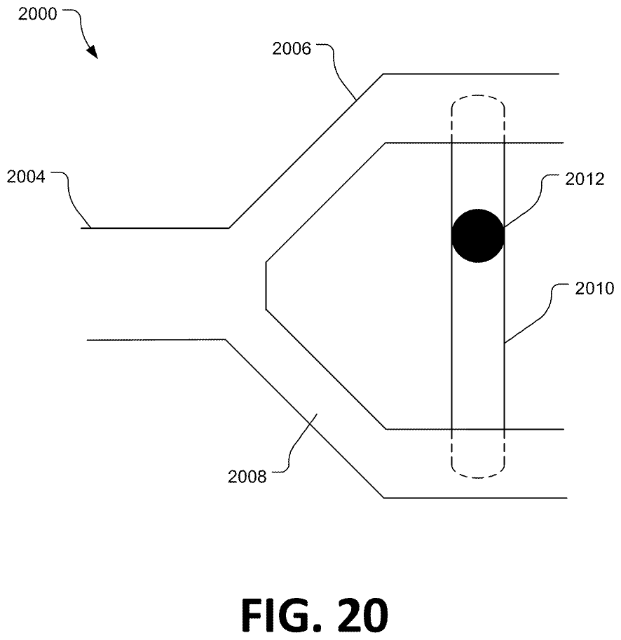

FIG. 20 depicts another example channel-selection system 2000. The system 2000 includes an inlet 2004, a first channel 2006, and a second channel 2008. The system 2000 also includes a track 2010 and a slidable element 2012, which may be a ball, disk, or other type of physical element. The slidable element may be slid along the track to compress or pinch either the first channel 2006 of the second channel 2008. For instance, when the slidable element 2012 is slid to one end of the track 2010, the second channel 2008 is compressed and the flow of gas through the second channel 2008 is blocked. When the slidable element 2012 is slid to the other end of the track, the first channel 2006 is compressed and the flow of gas through the first channel 2006 is blocked.

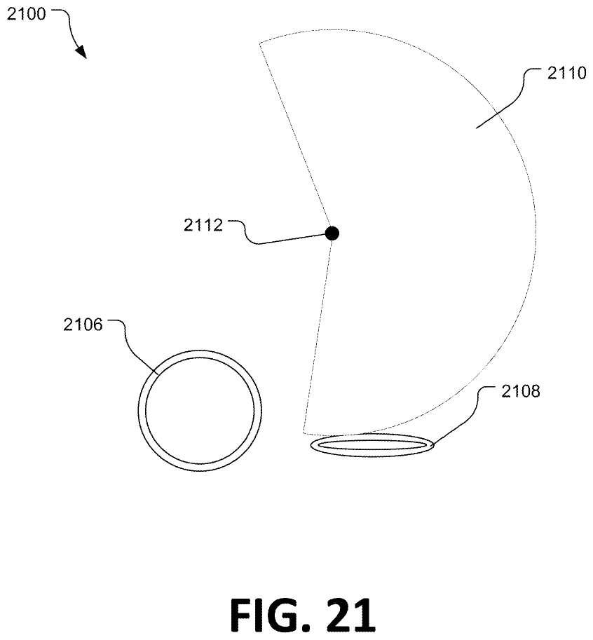

FIG. 21 depicts another example channel-selection system 2100. The system 2100 includes a first channel 2106 and a second channel 2108. The system 2100 also includes a swing clamp 2110 that may be rotated around a pivot point 2112. When the swing clamp 2110 is rotated to a first position, the swing clamp 2110 compresses the second channel 2108 and prevents gas flow through the second channel 2108. When the swing clamp is rotated to a second position, the swing clamp 2110 compresses the first channel 2106 and releases the compression of the second channel 2108. Accordingly, the swing clamp may be positioned to selectively control whether gas flows through the first channel 2106 or the second channel.

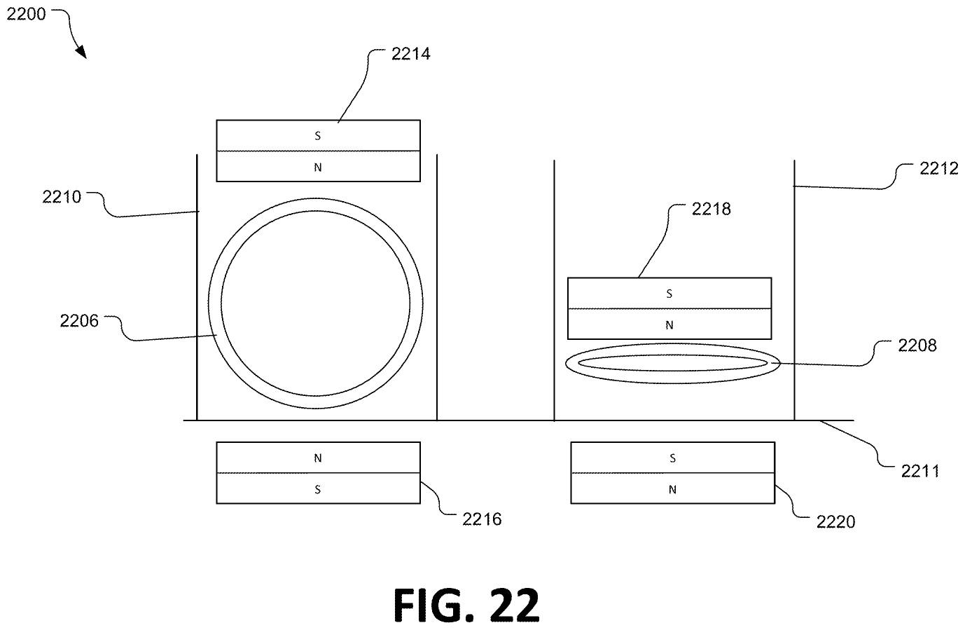

FIG. 22 depicts another example channel-selection system 2200. The system 2200 includes a first channel 2206 and a second channel 2208. The first channel 2206 is positioned within a first housing 2210, which may include two walls and a base 2211. The second channel 2208 is positioned within a second housing 2212, which may include two walls and a base 2211. In some examples, the first housing 2210 and the second housing 2212 may also share a common interior wall between the first channel 2206 and the second channel 2208.

The system 2200 also includes a first slidable magnet 2214, a second slidable magnet 2218, a first selection magnet 2216, and a second selection magnet 2220. The position of the first slidable magnet 2214 is controlled by the position of the first selection magnet 2216. The first selection magnet 2216 may be moved or manipulated by a clinician and/or the ventilator. When the first selection magnet 2216 is in a first position, the first selection magnet 2216 attracts the first slidable magnet 2214 and causes the first slidable magnet 2214 to slide towards the first selection magnet 2216 and compress the tubing of the first channel 2206. When the first selection magnet 2216 is in a second position, the first selection magnet 2216 repels the first slidable magnet 2214 and the first slidable magnet 2214 slides away from the first selection magnet 2216, which allows the first channel 2206 to open.

The position of the second slidable magnet 2218 is controlled by the position of the second selection magnet 2220. The second selection magnet 2220 may be moved or manipulated by a clinician and/or the ventilator. When the second selection magnet 2220 is in a first position, the second selection magnet 2220 attracts the second slidable magnet 2218 and causes the second slidable magnet 2218 to slide towards the second selection magnet 2220 and compress the tubing of the second channel 2208. When the second selection magnet 2220 is in a second position, the second selection magnet 2220 repels the second slidable magnet 2218 and the second slidable magnet 2218 slides away from the second selection magnet 2220, which allows the second channel 2208 to open.

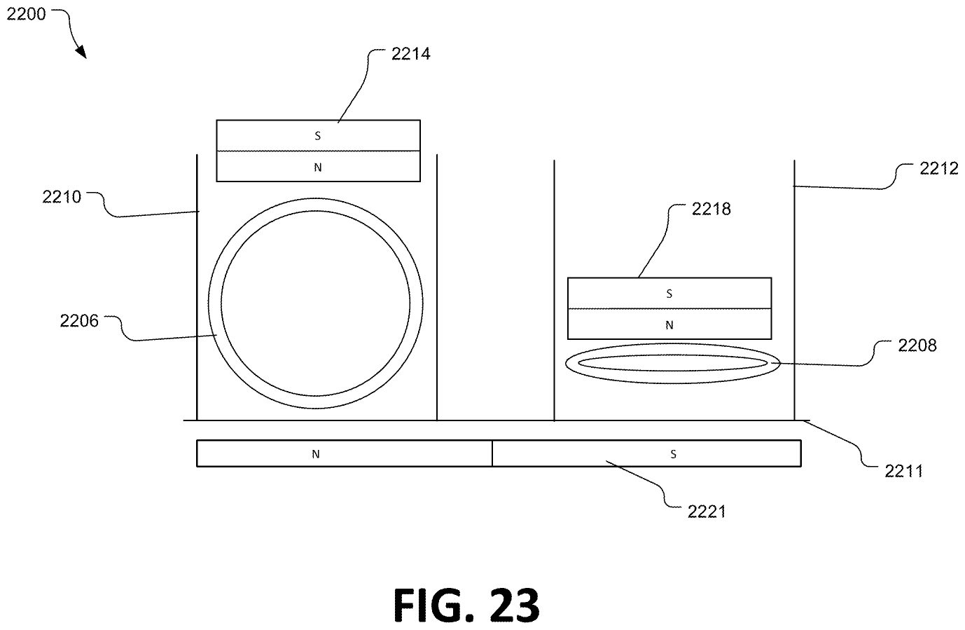

FIG. 23 depicts a variation of the example channel-selection system 2200 of FIG. 22. Instead of using a second slidable magnet 2218 and a second selection magnet 2220, a single selection magnet 2221 may be used to control the positions of the first slidable magnet 2214 and the second slidable magnet 2218. When the single selection magnet 2221 is in a first position, the single selection magnet 2221 attracts the second slidable magnet 2218 and repels the first slidable magnet 2214. When the single selection magnet 2221 is in a second position, the single selection magnet 2221, attracts the first slidable magnet 2214 and repels the second slidable magnet 2218.

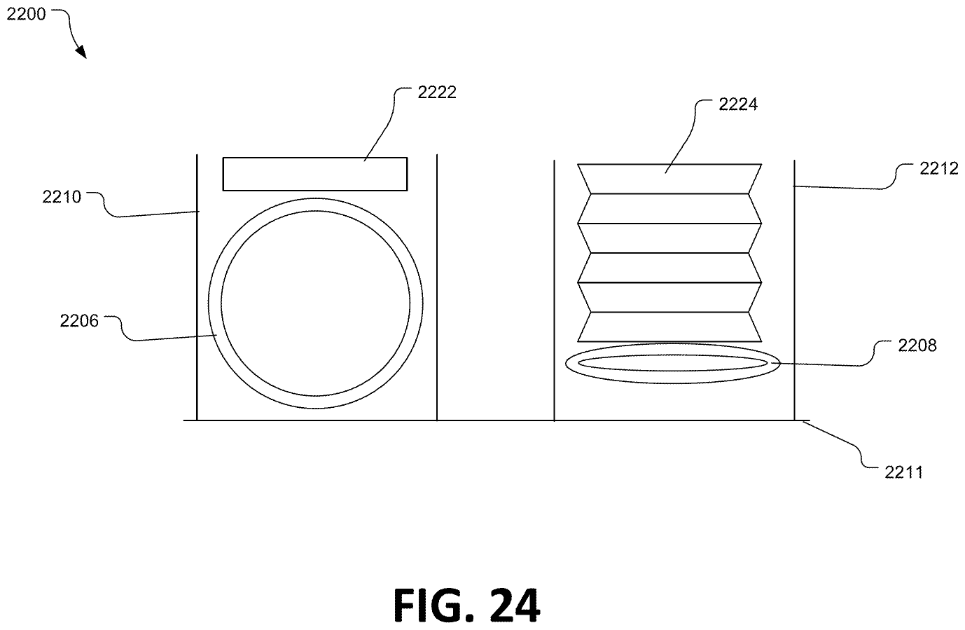

FIG. 24 depicts another variation of the example channel-selection system 2200 of FIG. 22. Instead of using magnetic forces to control the compression of the tubing of the first channel 2206 or the second channel 2208, the system 2200 in FIG. 24 uses pneumatic forces. For instance, the system 2200 uses a first expandable pneumatic component 2222 and a second expandable pneumatic component 2224. The expandable pneumatic components 2222, 2224 may include elements such as bellows, pistons, or the like that physically move in response to pneumatic forces. When a pneumatic force is applied to the first expandable pneumatic component 2222, the first expandable pneumatic component 2222 expands and compresses the tubing of the first channel 2206. Similarly, when a pneumatic force is applied to the second expandable pneumatic component 2224, the second expandable pneumatic component 2224 expands and compresses the tubing of the second channel 2208, as shown in FIG. 24. The pneumatic forces may be selectively applied or controlled by the ventilator and/or a clinician. The source of the pneumatic forces may include pressurized gases from compressed air that is frequently available from an air port (e.g., wall port) in medical facilities such as hospitals.

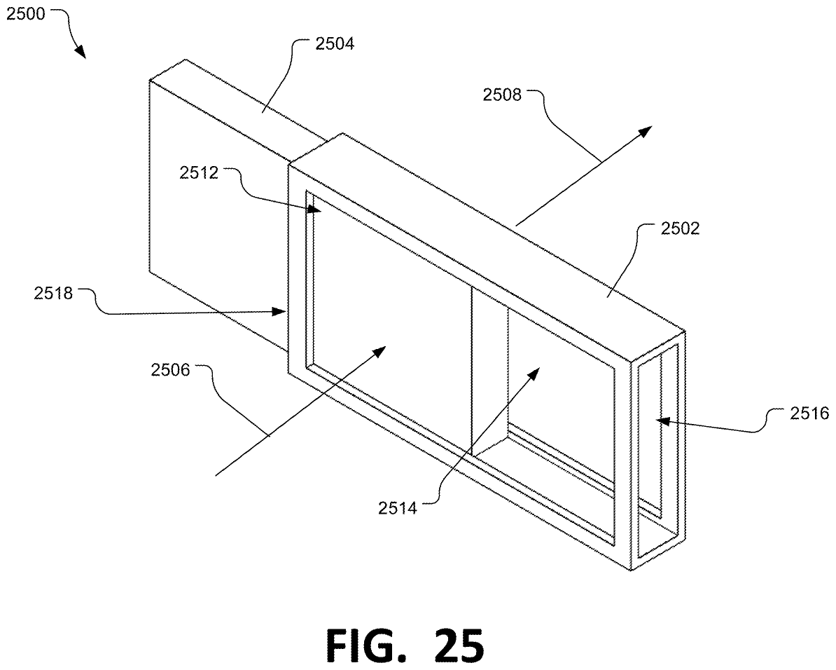

FIG. 25 depicts a perspective view of a replaceable filter system 2500. The replaceable filter system 2500 includes a frame 2502 and a first filter or filter media 2504. Unfiltered gases 2506 flow through the first filter media 2504, where they are filtered to form filtered gases 2508. The frame 2502 and the first filter media 2504 are configured such that the first filter media 2504 may slide through the frame 2502.

In the example depicted, the frame 2502 includes a front window 2512 facing the unfiltered gases 2506 and a rear window 2514 facing the filtered gases 2508. The windows or openings in the frame that allow for gas flow through the filter media in the frame 2502, such as the first filter media 2504 in the frame 2502. The frame 2502 also includes openings 2516, 2518 for slidably receiving new filter media. In the example depicted, the sides adjacent to the front and back sides (e.g., the left and right sides) both have openings through which the first filter media may slide. The opening through which new filters are received may be referred to as the filter-receiving opening 2516, and the opening through which the dirty filters are ejected may be referred to as the filter-ejection opening 2518. The sides with the openings 2516, 2518 may be referred to as non-gas exposed sides or lateral sides of the frame 2502. While the first filter media 2504 is shown in FIG. 25 as being only partially within the frame, during ventilation the first filter media 2504 fills the entire front window 2512 such that unfiltered gas 2506 does not flow through the frame 2502 without being filtered.

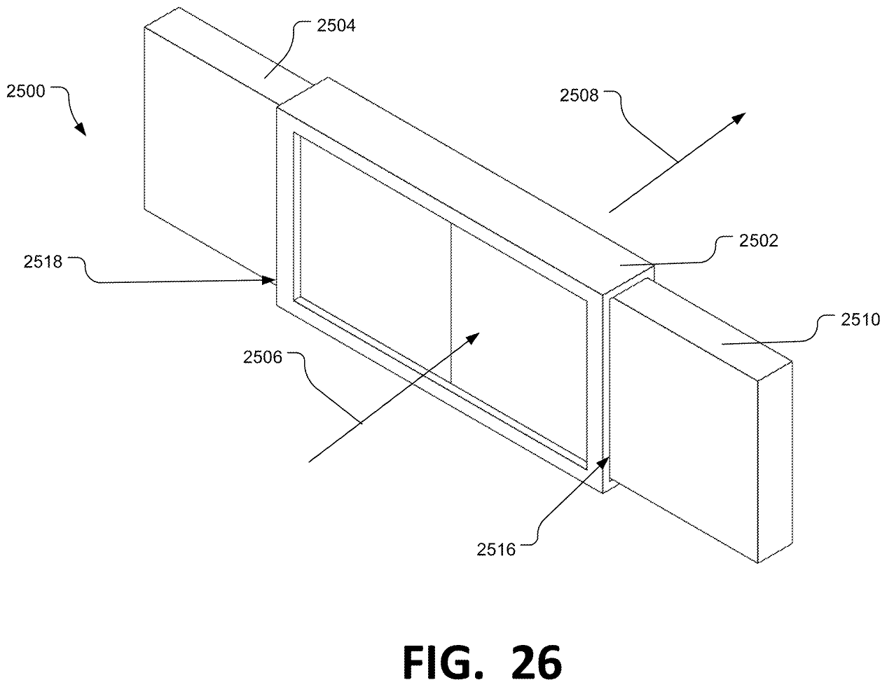

FIG. 26 depicts another perspective view of the replaceable filter system 2500 of FIG. 25. In FIG. 26, a second filter or filter media 2510 has been slid into the frame 2502 and is in contact with the first filter media 2504. During ventilation, the second filter media may be slid through the filter-receiving opening 2516 and remain in contact with the first filter media 2504 as the second filter media 2510 is slid into the frame 2502. For instance, there may be no gaps between the first filter media 2504 and the second filter media 2510 that would allow for unfiltered gas to flow through the frame 2502 without being filtered by either the first filter media 2504 or the second filter media 2510. Thus, as the new, or second, filter media 2510 is slid into the frame 2502, the first filter media 2504 is expelled out the filter-ejection opening all while ventilation is able to continue and the expiratory gases are still filtered.

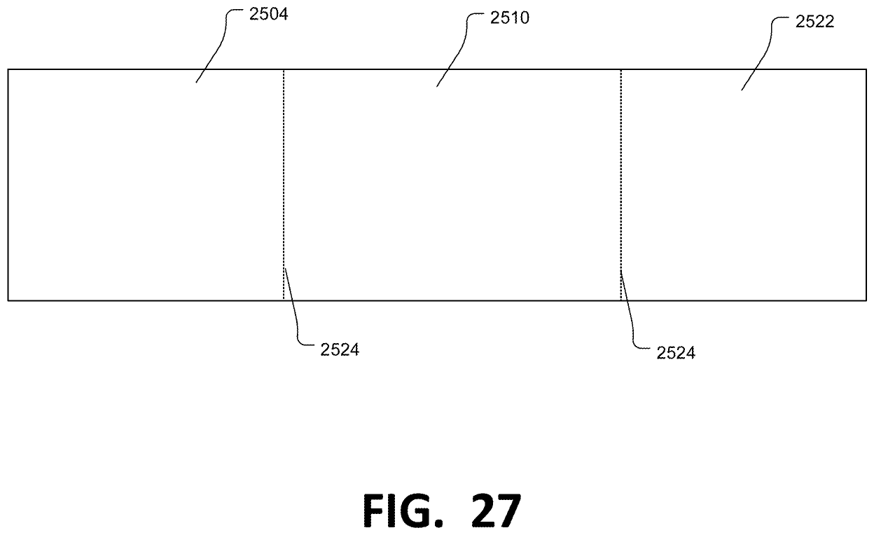

Each of the new filter media that are inserted into the frame 2502 may be individual components that are separate components. In other examples, there may be a plurality of connected filter media, such as a roll or sheet of filter media. FIG. 27 depicts such a plurality of connected filter media. In FIG. 27, the first filter media 2504 is connected to the second filter media 2510, which is in turn connected to a third filter media 2522. While only three filters or filter media are depicted, additional pieces of filter media may be attached as well. A perforated line 2524 is positioned between each piece of filter media. For instance, a perforated line 2524 is positioned between the first filter media 2504 and the second filter media 2510. Another perforated lines 2524 is also positioned between the second filter media and the third filter media 2522. The perforated lines 2524 increase the ease and accuracy with which the respective filter media may be broken or torn apart from one another when ejected from the frame 2502. For instance, the sheet or roll of connected filter media may be fed into the frame, and as a piece of filter media is ejected from the filter-ejection opening 2518, it may be torn or broken off from the remainder of the roll or sheet and be disposed. In other examples, the sheet or roll is one continuous sheet of filter media that is fed through the frame. As one portion of the filter media degrades, the filter sheet is further fed through the frame such that the unfiltered gases 2506 are exposed to portion of the sheet or roll that has clean filter media.

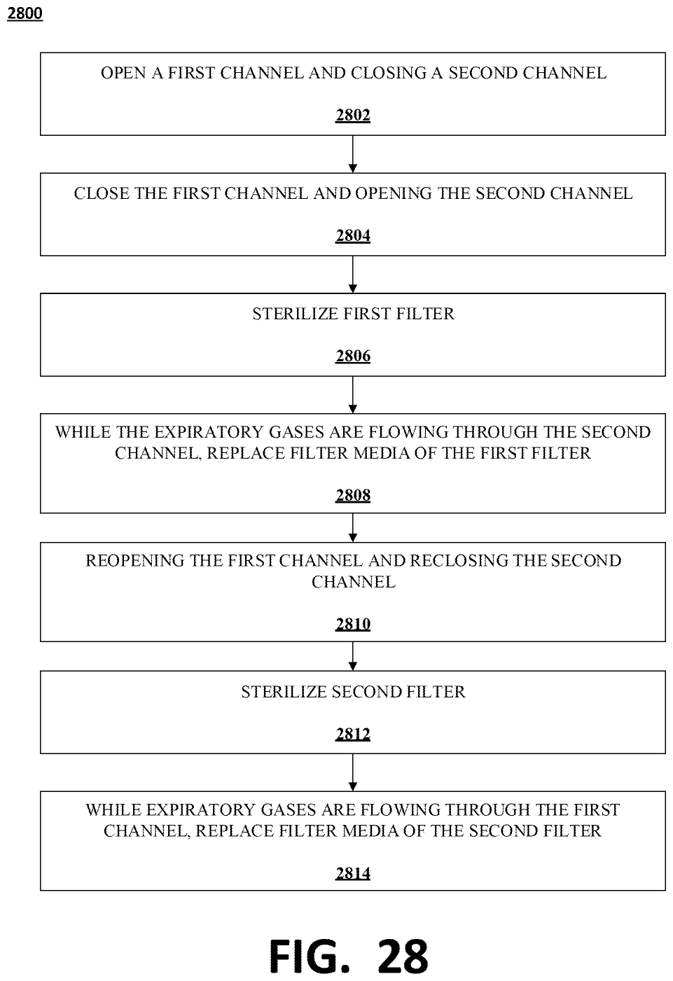

FIG. 28 depicts an example method 2800 for selecting a filtration channel. At operation 2802, a first channel of a dual-channel filtration system is opened a second channel of the system is closed, such that expiratory gases flow through a first filter coupled to the first channel. At operation 2804, the first channel is closed and the second channel is opened, such that the expiratory gases flow through a second filter coupled to the second channel. Opening and closing of the respective channels may be performed using any of the various channel-selection mechanisms and procedures discussed above. The opening and closing of the various channels may also be performed manually or controlled by the ventilator.

At operation 2806, the first filter is sterilized. Sterilization of the first filter may occur while the gases are flowing through the first channel, or subsequent to the cessation of the gas flow through the first channel. The sterilization of the first filter may be accomplished through the use of a sterilization system, such as the UV sterilization system discussed above.

At operation 2808, while the expiratory gases are flowing through the second channel and, in some examples, after sterilization of the first filter has occurred, the filter media of the first filter is replaced. Replacement of the filter media may include opening of a housing that houses the first filter and the filter media. Then, while the housing is opened, the filter media may be manually removed and replaced with clean filter media.

At operation 2810, the first channel is reopened and the second channel is reclosed such that the expiratory gases flow through the first channel and the first filter with the replaced filter media. Operation 2810 may be performed some time after operation 2808, such as when performance of the second filter has degraded and needs replacing. At operation 2812, the second filter is sterilized. Sterilization of the second filter may be accomplished through the same or substantially similar procedure as sterilization of the first filter. In some examples, the first filter and the second filter may share a common sterilization system. In other examples, separate sterilization systems are used for the first filter and the second filter. At operation 2814, while the expiratory gases are flowing through the first filter channel, the filter media of the second filter is replaced.

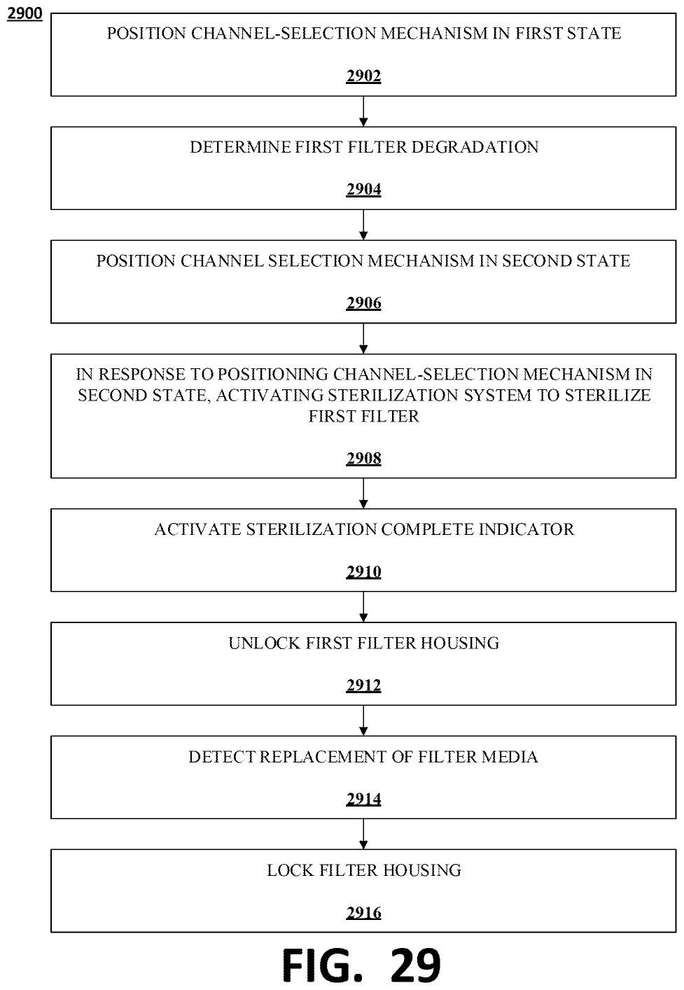

FIG. 29 depicts an example method 2900 for replacing an expiratory filter. One or more of the operations of method 2900 may be performed by a ventilator and/or a controller for the dual-channel filtration system. At operation 2902, a channel-selection mechanism is positioned in a first state to allow for expiratory gases to flow through a first channel and a first filter and to prevent the expiratory gases from flowing through a second channel and a second filter.

At operation 2904, a first filter degradation is determined or detected. Determination of filter degradation may be based on an amount of time that has passed since the first filter was replaced, a duration of ventilation that has occurred since the first filter was replaced, a type of ventilation being delivered, a humidification setting of for the ventilation being delivered, a type or condition of the patient (e.g., some patients may be more prone to expelling mucus or other liquids that may clog the filter more quickly), and/or a measurement from sensors on the ventilator, patient circuit, and/or the filter housing itself. For example, if an amount of time since the filter has been replaced passes a filter-duration threshold, the filter degradation may be determined. As another example, an increase in a pressure differential across the filter may also indicate degradation of the filter, such as clogging of the filter. The pressure differential may be identified by seeing an increase in pressure on the expiratory side of the patient circuit despite no changes to the ventilation settings. Such a pressure increase, however, may be attributed to other potential causes in addition to or alternatively to the filter. To assist in identifying a degraded filter as the root cause, one or more pressure sensors may be located in the filter housing to identify a changing pressure differential across the filter. When a filter degradation has been detected, an alert or indicator may be activated to indicate that the filter needs replacement. Such an indicator may be a visual indicator (e.g., light) on the filter housing. The indicator may also or alternatively be displayed on the display of the ventilator.

At operation 2906, the channel-selection mechanism is positioned in a second state to allow for expiratory gases to flow through the second channel and the second filter and to prevent the expiratory gases from flowing through the first channel and the first filter. Operation 2906 may be automatically performed in response to determination of a filter degradation in operation 2904. For instance, based on the determination that the first filter has degraded, the ventilator may automatically position the channel-selection mechanism in the second state. In other examples, the operation 2906 may be performed in response to a selection received from the clinician. For instance, the clinician may select a displayed option on the ventilator to cause the channel-selection mechanism to change states.

At operation 2908, in response to or based on the channel-selection mechanism being positioned in the second state, a sterilization system is activated to sterilize the first filter. While the filter is being sterilized, an indicator may be displayed or activated. The indicator may be visual or audible. The indicator may be displayed or emitted from the ventilator or a component of the dual-channel filtration system. In other examples, the sterilization of the first filter may be performed in response to receiving a selection from a clinician rather than automatically in response to the channel-selection mechanism changing states.

Once the sterilization process has been completed, a sterilization-complete indicator 2910 may be activated. The sterilization-complete indicator may be an audible or a visual indicator to indicate the completion of the sterilization process. The indicator may be emitted or displayed on the ventilator and/or on the first filter housing itself.

At operation 2912, upon completion of the sterilization process (or shortly thereafter), the first filter housing that houses the first filter is unlocked such that a clinician may access the first filter to replace the filter media. The filter housing may generally be in a locked state during ventilation and during sterilization. Locking the filter housing provides for additional safety measures. For instance, opening of the filter housing during ventilation may allow for pathogens to escape and pressure loss in the patient circuit to occur. Opening of the filter housing during sterilization may expose a clinician to UV light. Thus, the filter housing may be unlocked after the sterilization process has completed and no gas is flowing through the first filter housing.

At operation 2914, a replacement of the filter media is detected. Such a detection may occur automatically via position sensors that identify a removal and a replacement of the filter media. For instance, the filter housing may include an optical-gate sensor that detects the presence of the filter and/or filter media. In some examples, the optical-gate sensor signal may also be used changing the channel-selection mechanism from one state to another. For example, the sensor indicates no filter is installed in a particular channel, the ventilator may not change the channel-selection mechanism to direct gas towards the channel without the filter.

In other examples, the replacement may be based on input received from the clinician. For instance, the clinician may select an option on the ventilator that confirms that the replacement has occurred. Based on detecting that the filter media has been replaced, the first filter housing is locked again at operation 2916. The method 2900 may then repeat for the second filter housing as the expiratory gas flows through the second filter.

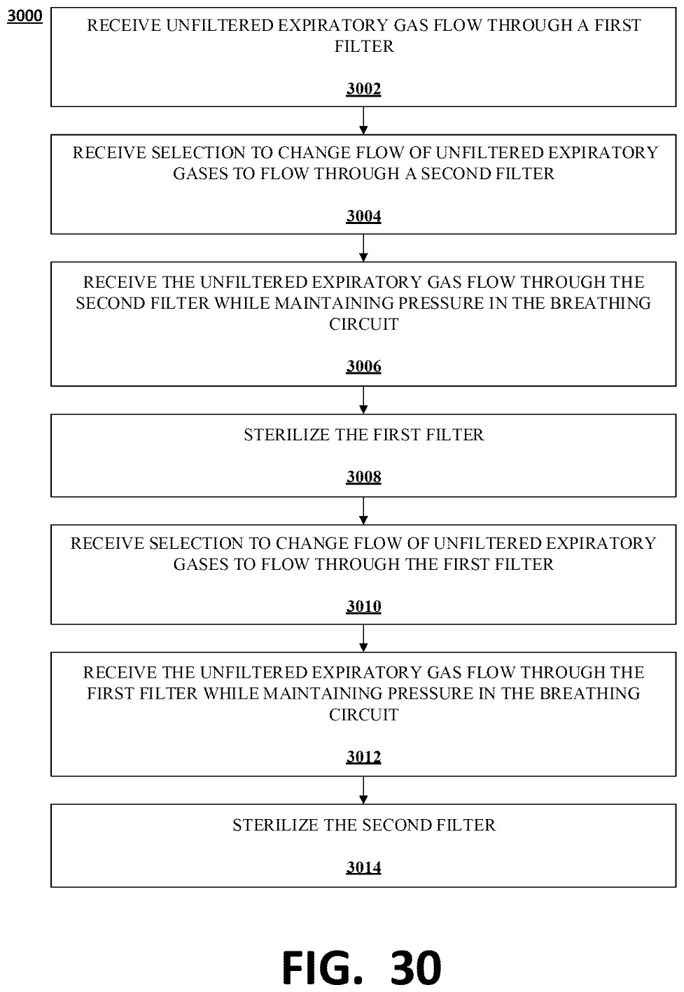

FIG. 30 depicts an example method 3000 for filtering expiratory gases while maintaining pressure in the breathing circuit. One or more of the operations of method 3000 may be performed by a ventilator and/or a controller for the filtration system.

At operation 3002, an unfiltered expiratory gas flow is received through a first filter. For example, the unfiltered expiratory gas flow may be received via an inlet of the filtration system and directed through a first channel that includes the first filter. As the unfiltered expiratory gas flows through the first filter, the unfiltered expiratory gas is filtered to form filtered expiratory gas. The filtered expiratory gas may then flow out the outlet of the filtration system.

At operation 3004, a selection to change the flow of the unfiltered expiratory gases through a second filter is received. The selection may be a manual selection, such as from a clinician, or an automatic signal generated from the filtration system and/or the ventilator. Based on receiving the selection, the filtration system adjusts such that the unfiltered expiratory gas flows through the second filter, which may be positioned in a second channel of the filtration system. For example, any of the selection mechanisms discussed herein may be adjusted to change the expiratory gas flow from the first channel to the second channel.

At operation 3006, subsequent to receiving the selection in operation 3004, the unfiltered expiratory gas flow is received through the second filter while maintaining pressure in the breathing circuit. For instance, the continuity of the breathing circuit is maintained while the unfiltered expiratory gas flow is redirected from the first channel to the second channel. In some examples, as the flow is transitioning from the first channel to the second channel, the expiratory gases may flow through both the first channel and the second channel. Once the transition is completed, the expiratory gas may flow through the second channel, but not the first channel, which allows for the first filter to be replaced without depressurizing the circuit. Thus, during the transition, and subsequent to the transition, the continuity and pressure of the breathing circuit is maintained. As a result, ventilation of the patient is able to continue in an uninterrupted manner while the first filter of the filtration system is replaced. For instance, pressures, such as PEEP, are maintained during, and after, the transition from the first channel to the second channel. While the unfiltered expiratory gases are flowing through the second filter, the second filter filters the unfiltered expiratory gases to form filtered expiratory gases. The filtered expiratory gases may then flow out the outlet of the filtration system.

At operation 3008, the first filter may be sterilized while the expiratory gases are flowing through the second filter and not the first filter. Operation 3008 may include multiple sterilization steps or operations discussed herein, such as the operations set forth in method 2900 discussed above with reference to FIG. 29.

At operation 3010, a selection to a selection to change the flow of the unfiltered expiratory gases through the first filter, instead of the second filter, is received. The selection may be substantially similar as the selection received in operation 3004, but the selection in operation 3010 is to change expiratory gas flow from the second filter to the first filter.

At operation 3012, subsequent to receiving the selection in operation 3010, the unfiltered expiratory gas flow is received through the first filter while maintaining pressure in the breathing circuit. The transition from flow through the second filter to the first filter may be performed similarly as the transition from flow through the first filter to the second filter, but the transition is in the opposite direction. For instance, as flow is redirected from the second channel to the first channel, continuity of the breathing circuit and the gas pressures therein are maintained during and after the transition. At operation 3014, the second filter is sterilized while the expiratory gases are flowing through the first filter and not the second filter. The sterilization operation 3014 may be similar to the sterilization operation 3008.

While method 3000 discusses redirecting expiratory gas flow from a first filter to a second filter and then back to the first filter, in other examples there may be more than two filters and channels. In such examples, the expiratory gas flow may be first directed through the first filter and the first channel, then directed through the second channel and the second filter, and then subsequently to a third channel and a third filter rather than back to the first filter, etc. In such examples, continuity and pressure of the breathing circuit may still be maintained through all the transitions and during the replacement of the corresponding filters.

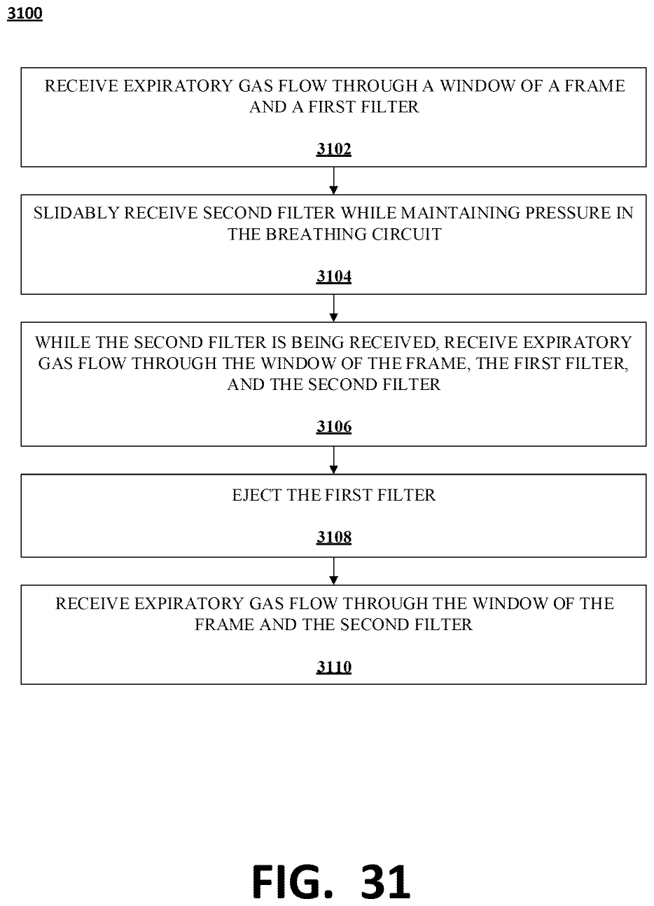

FIG. 31 depicts an example method 3100 for filtering expiratory gases while maintaining pressure in the breathing circuit. At operation 3102, expiratory gases are received through a window of a frame and a first filter disposed within the frame. The first filter filters the expiratory gases that flow through the window.

At operation 3104, a second filter is slidably received via a filter-receiving opening of the frame. As the second filter is slidably received, the second filter contacts the first filter and the first filter begins to slide out of the frame via a filter-ejection opening of the frame. At operation 3106, while the second filter is being received and prior to the first filter being fully ejected from the frame, the expiratory gas flow is received through the window of the frame, the first filter, and the second filter. For instance, one portion of the expiratory gases may be filtered by the first filter and another portion of the expiratory gases may be filtered by the second filter. The pressure in the breathing circuit may be maintained during this transition process. For example, a first seal may be provided between the first filter and the filter-ejection opening and a second seal may be provided between the second filter and the filter-receiving opening. In addition, because the second filter is in contact with the first filter during the transition, the expiratory gases are continuously filtered during the transition process.

At operation 3108, once the second filter is fully received into the frame, the first filter is ejected from the frame via the frame-ejection opening. Once ejected, the first filter may be disposed or recycled. At operation 3110, subsequent to the first filter being ejected, the expiratory gas flow is received through the window of the frame and the second filter such that the second filter filters the expiratory gases.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A ventilation system, comprising:
an inspiratory limb that carries breathing gases from a medical ventilator towards a patient;
an expiratory limb that carries expiratory gases from the patient towards the medical ventilator; and
a dual-channel expiratory filtration system coupled to the expiratory limb and configured to receive unfiltered expiratory gases carried by the expiratory limb, the dual-channel expiratory filtration system comprising:
an inlet that receives the unfiltered expiratory gases from the expiratory limb;
a first channel, pneumatically coupled to the inlet, including a first filter that filters the unfiltered expiratory gases to form filtered expiratory gases;
a second channel, pneumatically coupled to the inlet, including a second filter that filters the unfiltered expiratory gases to form filtered expiratory gases;
at least one valve, wherein when the valve is in a first state, the unfiltered expiratory gases from the inlet flow through the first channel, and when the valve is in a second state, the unfiltered expiratory gases from the inlet flow through the second channel; and
an outlet, pneumatically coupled to the first channel and the second channel, that carries the filtered expiratory gases towards the ventilator.

2. The system of claim 1, wherein the at least one valve includes a first valve positioned in the first channel and a second valve positioned in the second channel.

3. The system of claim 1, wherein the at least one valve is a rotatable valve that is:
coupled to the inlet;
selectively coupled to the first channel when the rotatable valve is in the first state; and
selectively coupled to the second channel when the rotatable valve is in the second state.

4. The system of claim 1, wherein the valve is controlled by the ventilator.

5. The system of claim 1, wherein the valve is configured to be controlled manually by a clinician.

6. The ventilation system of claim 1, further comprising:
a first check valve in a first post-filter segment of the first channel; and
a second check valve in a second post-filter segment of the second channel.

7. The ventilation system of claim 1, further comprising a sterilization system configured to sterilize the first filter.

8. A method for filtering expiratory gases while maintaining pressure in a breathing circuit, the method comprising:
receiving an unfiltered expiratory gas flow through a first filter positioned in an expiratory limb of breathing circuit;
receiving a selection to change the unfiltered expiratory gases to flow through a second filter positioned in the expiratory limb of the breathing circuit; and
subsequent to receiving the selection, receiving the unfiltered expiratory gas flow through the second filter, but not the first filter, while maintaining pressure in the breathing circuit.

9. The method of claim 8, further comprising, while the unfiltered expiratory gas flow is received through the second filter, sterilizing the first filter.

10. The method of claim 8, further comprising:
receiving a selection to change the unfiltered expiratory gases to flow through the first filter positioned in the expiratory limb of the breathing circuit; and
receiving the unfiltered expiratory gas flow through the first filter, but not the second filter, while maintaining pressure in the breathing circuit.

11. The method of claim 10, further comprising, while the unfiltered expiratory gas flow is received through the first filter, sterilizing the second filter.

12. The method of claim 8, wherein the first filter is positioned in a first channel of a filtration system in the expiratory limb, and the second filter is positioned in a second channel of the filtration system.

13. A method for replacing expiratory filters of a ventilation system without breaking a breathing circuit, the method comprising:
opening a first channel and closing a second channel such that expiratory gases flow through a first filter coupled to the first channel;
closing the first channel and opening the second channel such that the expiratory gases flow through a second filter coupled to the second channel; and
while the expiratory gases are flowing through the second channel, replacing filter media of the first filter while maintaining pressure in the breathing circuit.

14. The method of claim 13, wherein the first channel and the second channel are both coupled to an inlet and an outlet.

15. The method of claim 13, further comprising, subsequent to replacing the filter media of the first filter:
reopening the first channel and reclosing the second channel such that the expiratory gases flow through first channel and the first filter; and
while the expiratory gases are flowing through the first channel, replacing filter media of the second filter.

16. The method of claim 13, further comprising subsequent to closing the first channel and opening the second channel, and prior to replacing the filter media of the first filter, sterilizing the first filter.

17. The method of claim 13, wherein opening the first channel and closing the second channel comprises rotating a rotatable valve.

18. The method of claim 17, wherein opening the second channel and closing the first channel comprises rotating the rotatable valve.

19. The method of claim 13, wherein closing the second channel comprises compressing tubing of the second channel.

20. The method of claim 13, wherein opening the first channel and closing the second channel comprises rotating a rotatable valve.
